(19) United States
(12) Patent Application Publication
(10) Pub. No.: US 2015/0283047 A1
(43) Pub. Date: Oct. 8, 2015

(54) TEXTILE-SPARING ANTIPERSPIRANT SPRAY WITH METHANESULPHONIC ACID

(71) Applicant: Henkel AG & Co. KGaA, Duesseldorf (DE)

(72) Inventors: Bernhard Banowski, Duesseldorf (DE); Marcus Claas, Hilden (DE)

(21) Appl. No.: 14/742,769

(22) Filed: Jun. 18, 2015

Related U.S. Application Data
(63) Continuation of application No. PCT/EP2013/076651, filed on Dec. 16, 2013.

(30) Foreign Application Priority Data
Dec. 21, 2012 (DE) 10 2012 224 142.6

(51) Int. Cl. A61K 8/26; A61K 8/06; A61Q 15/00; A61K 8/46

(52) U.S. Cl. CPC A61K 8/26; A61K 8/466; A61K 8/062; A61Q 15/00; A61K 2800/30

(57) ABSTRACT

The invention concerns cosmetic antiperspirants for spray application containing methanesulphonic acid and/or at least one physiologically compatible salt of methanesulphonic acid which soils textiles to a lesser degree than known antiperspirants.

### FIELD OF THE INVENTION

The present invention generally relates to cosmetic antiperspirant sprays which soil textiles to a lesser degree than known antiperspirants.

### BACKGROUND OF THE INVENTION

Washing, cleansing, and hygiene of the body are basic human needs, and modern industry constantly attempts to meet these human needs in many ways. The constant elimination or at least reduction of body odor is particularly important for daily hygiene. In the prior art, numerous specific deodorant or perspiration-inhibiting body care products are known which were developed for application in body regions with a high density of sweat glands, particularly in the armpit region. These are prepared in the most varied forms of application, for example, as a powder, in the form of a stick, as an aerosol spray, pump spray, liquid and gelatinous roll-on application, cream, gel, and as an impregnated flexible substrate (deodorant wipes).

In any case, cosmetic antiperspirants contain at least one perspiration-inhibiting salt. They frequently further contain at least one oil or a fatty substance and, in any case, always an odorous substance component or a perfume.

The regular use of antiperspirants can result in clearly visible colored textile soiling. Frequently, these are yellow stains which also cannot be removed by intensive washing. The formation of stains is based on a complex interaction of formulation components, perspiration, and the detergent used. Initially, insoluble aluminum compounds are probably formed on and within the fiber. In general, the yellow coloration occurs with a time delay and is at least to some extent caused by oxidation of unsaturated fatty acids which are present as insoluble aluminum salts. Unfortunately, different factors can interact here in an unexpected manner and, depending on the selection of the perfume oil, detergent and depending on the individual amount of perspiration and its composition, distinct yellow stains can from on textiles.

Due to the interaction of detergents and antiperspirant active substances, insoluble compounds are formed which can attach themselves to textiles. These insoluble compounds form white, hard residues which frequently become visible on the fabric only after several soiling and wash cycles. These white residues are not soluble in water and can also not be removed using a standard washing method. They are particularly apparent on light- or dark-colored textiles. A skillful selection of additives results in a significantly decreased or delayed formation of these insoluble deposits.

For masking white residues on dark textiles, e.g. through transfer of the products from the skin onto a textile when dressing, cosmetic oils or polyols are used. These masking agents can also attach themselves to a textile. Depending on the chemical composition, these masking agents can only be removed to some extent, if at all, using a standard washing process. The hydrophobic masking agent accumulates on the textile and results in a dark, greasy/oily stain which, among others, can also change the haptics of the textiles in the soiled region. A skillful selection of additives results in a significantly decreased or delayed formation of these greasy/oily dark soilings.

Therefore, there is a need for antiperspirant formulations which can reliably prevent the formation of yellow, white and/or greasy stains.

In order to protect textiles from such permanent soilings, various ingredients are added in the prior art. A frequently used additive are surfactants, as disclosed, for example in WO 2010/097205 A2. The selection of the oil components can also decrease or even increase the textile soiling, see, for example U.S. Pat. No. 5,925,338, U.S. Pat. No. 4,511,554, or U.S. Pat. No. 3,974,270.

The present invention addresses the problem of providing cosmetic antiperspirant sprays which contain a perspiration-inhibiting zirconium-free aluminum salt and do not, or only to a greatly reduced extent, result in permanent textile discolorations.

Surprisingly, it was now found that an additive of methanesulphonic acid and/or at least one physiologically compatible salt of methanesulphonic acid solves the stated problems very well and is preeminently suitable as discoloration inhibitor for use in cosmetic antiperspirants.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Cosmetic product, consisting of i) a packaging, selected from a pump spray container, a squeeze container, and a spray can containing at least one propellant; and ii) a perspiration-inhibiting cosmetic agent contained therein for spray application, containing, in a cosmetically compatible carrier, at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the agent without taking into account an optionally present propellant; and additionally methanesulphonic acid of the following formula (MS-1)

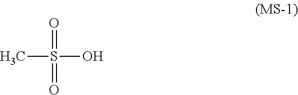

or at least one physiologically compatible salt of methanesulphonic acid.

Use of methanesulphonic acid of the formula (MS-1)

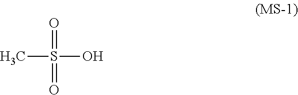

and/or at least one physiologically compatible salt of methanesulphonic acid in a cosmetically compatible carrier, containing at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the propellant-free agent, for reducing or preventing textile discolorations and/or textile stains.

Method for preventing and/or reducing textile discolorations and/or textile stains, wherein the method comprises the following method steps: (a) producing a perspiration-inhibiting cosmetic agent by mixing at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the agent, with a cosmetically compatible carrier and with methanesulphonic acid of the following formula (MS-1)

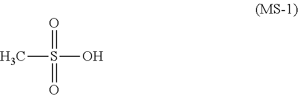

or at least one physiologically compatible salt of methanesulphonic acid, wherein methanesulphonic acid or the salt(s) thereof is/are preferably contained in a total amount of 0.05 to 5 wt. %, more preferably 0.1 to 3 wt. %, particularly preferably 0.5 to 2.5 wt. %, extraordinarily preferably 1 to 2 wt. %, wherein the specifications in wt. % each refer to the total weight of the propellant-free agent; (b) filling of the agent in a packaging selected from a pump spray container, a squeeze container, and a spray can containing at least one propellant; (c) applying the perspiration-inhibiting cosmetic agent to the skin, particularly to the skin of the armpits; (d) wearing a textile garment over the treated skin; and (e) washing the textile garment, particularly repeatedly washing the textile garment, wherein no, or reduced, textile discolorations and/or textile stains occur after washing, particularly after repeated washing.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present application is a cosmetic product consisting of
i) a packaging selected from a pump spray container, a squeeze container, and a spray can containing at least one propellant, and
ii) a perspiration-inhibiting cosmetic agent contained therein for spray application, containing, in a cosmetically compatible carrier,
a) at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the agent without taking into account an optionally present propellant, and additionally
b) methanesulphonic acid of the following formula (MS-1)

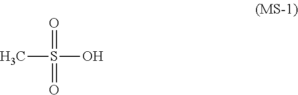

and/or at least one physiologically compatible salt of methanesulphonic acid.

A further subject matter of the present application is the use of methanesulphonic acid of the following formula (MS-1)

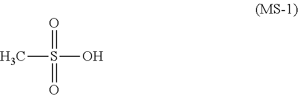

in a perspiration-inhibiting cosmetic agent, containing in a cosmetically compatible carrier at least one zirconium-free perspiration-inhibiting aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP)), free of water of crystallization and free of ligands, in the composition, for reducing or preventing textile discolorations and/or textile stains, wherein the specifications in wt. % each refer to the total weight of the agent.

What is stated below concerning the agents according to the invention applies, mutatis mutandis, to the preferred embodiments of the use according to the invention.

Preferred agents according to the invention are characterized in that the physiologically compatible salts of methanesulphonic acid are selected from the sodium, potassium, magnesium, calcium, manganese, zinc, and aluminum salts of methanesulphonic acid. Methanesulphonic acid itself is extraordinarily preferred.

Further preferred agents according to the invention are characterized in that they contain methanesulphonic acid and/or at least one physiologically compatible salt thereof in a total amount of 0.05 to 5 wt. %, preferably 0.1 to 3 wt. %, particularly preferably 0.5 to 2.5 wt. %, extraordinarily preferably 1 to 2 wt. %, in each case based on the total weight of the agent without taking into account an optionally present propellant.

At this point, it should be noted that, unless specified otherwise, the specification in wt. % in each case refers to the total weight of the agent according to the invention without taking into account an optionally present propellant.

In the present application, the terms “agent according to the invention” and “composition according to the invention” are used synonymously.

“Normal conditions” in terms of the present application are a temperature of 20° C. and a pressure of 1013 mbar. Melting point specifications also refer to a pressure of 1013 mbar.

The agents according to the invention contain a cosmetically compatible carrier. According to the invention, the cosmetically compatible carrier is preferably liquid under normal conditions (20° C., 1013 mbar). Further cosmetically compatible carriers preferred according to the invention comprise at least one cosmetic oil which is not an odorous substance and not an essential oil.

The cosmetic oils, which are liquid under normal conditions, are immiscible with water.

Insofar as a cosmetic oil is mentioned in the present application, it always refers to a cosmetic oil which is not an odorous substance and not an essential oil, is liquid under normal conditions and immiscible with water.

In a first preferred embodiment of the invention, the agent according to the invention contains zero to a maximum of 10 wt % free water, preferably zero to a maximum of 5 wt. % free water. The content of water of crystallization, hydration water or similarly molecularly bonded water contained in the components used, particularly in the perspiration-inhibiting active substances, is, in terms of the present application, not free water and thus not be taken into consideration for the calculation of the water amount.

Surprisingly, it was found that the textile discoloration-reducing or -preventing effect of methanesulphonic acid and/or at least one physiologically compatible salt thereof emerges particularly well in agents which contain 15 to 96 wt. % free water, based on the total weight of the agent according to the invention, without taking into account an optionally present propellant.

In a particularly preferred embodiment of the invention, the agent according to the invention thus contains free water in a total amount of 15 to 96 wt. %, preferably 25 to 80 wt. %, particularly preferably 30 to 70 wt. %, extraordinarily preferably 40 to 60 wt. %, in each case based on the total weight of the agent according to the invention without taking into account an optionally present propellant.

### Antiperspirant Active Substances

As antiperspirant active substance, the compositions according to the invention contain at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the composition, without taking into account an optionally present propellant.

The perspiration-inhibiting aluminum salts are preferably selected from the water-soluble astringent inorganic and organic salts of aluminum According to the invention, aluminosilicates and zeolites are not included among the antiperspirant active substances. According to the invention, water-solubility refers to a solubility of at least 3 wt. % at 20° C., i.e. amounts of at least 3 g of the antiperspirant active substance are soluble in 97 g water at 20° C.

Particularly preferred antiperspirant active substances are selected from aluminum chlorohydrate, particularly aluminum chlorohydrate of the general formula [Al₂(OH)₅Cl.1-6 H₂O]ₙ, preferably [Al₂(OH)₅Cl.2-3 H₂O]ₙ, which can be present in non-activated or activated (depolymerized) form, and aluminum chlorohydrate of the general formula [Al₂(OH)₄Cl₂.1-6 H₂O]ₙ, preferably [Al₂(OH)₄Cl₂.2-3 H₂O]ₙ, which can be present in non-activated or activated (depolymerized) form.

For example, the manufacture of preferred antiperspirant active substances is disclosed in U.S. Pat. No. 3,887,692, U.S. Pat. No. 3,904,741, U.S. Pat. No. 4,359,456, GB 2048229, and GB 1347950.

Further preferred are aluminum sesquichlorohydrate, aluminum dichlorohydrate, aluminum chlorohydrex propylene glycol (PG) or aluminum chlorohydrex polyethylene glycol (PEG), aluminum glycol complexes, e.g. aluminum or aluminum zirconium propylene glycol complexes, aluminum sesquichlorohydrex PG, or aluminum sesquichlorohydrex PEG, aluminum PG dichlorohydrex, or aluminum PEG dichlorohydrex, aluminum hydroxide, potassium aluminum sulfate (KAl(SO₄)₂.12 H₂O, alum), aluminum undecylenoyl collagen amino acid, sodium aluminum lactate+aluminum sulfate, sodium aluminum chlorohydroxylactate, aluminum bromohydrate, aluminum chloride, the aluminum salts of lipoamino acids, aluminum sulfate, aluminum lactate, aluminum chlorohydroxy allantoinate, and sodium aluminum chlorohydroxylactate.

Antiperspirant active substances particularly preferred according to the invention are selected from so-called “activated” aluminum salts, also called antiperspirant active substances “with enhanced activity.” Such active substances are known in the prior art and also commercially available. Their manufacture is disclosed, for example in GB 2048229, U.S. Pat. No. 4,775,528, and U.S. Pat. No. 6,010,688. In general, activated aluminum salts are produced through heat treatment of a relatively diluted solution of the salt (e.g. approximately 10 wt. % salt) in order to increase their HPLC peak 4 to peak 3 area ratio. The activated salt can subsequently be dried, particularly spray-dried, to form a powder. In addition to spray-drying, e.g., drum drying is also suitable.

Activated aluminum salts typically have an HPLC peak 4 to peak 3 area ratio of at least 0.4, preferably at least 0.7, and particularly preferably at least 0.9, wherein at least 70% of the aluminum is to be associated with these peaks.

Activated aluminum salts do not necessarily have to be used as spray-dried powder. Perspiration-inhibiting active substances also preferred according to the invention are non-aqueous solutions or solubilisates of an activated perspiration-inhibiting aluminum salt, for example, according to U.S. Pat. No. 6,010,688, which are stabilized against the loss of activation, i.e. against the rapid decrease of the HPLC peak 4 to peak 3 area ratio of the salt, by the addition of an effective amount of a polyvalent alcohol having 3 to 6 carbon atoms and 3 to 6 hydroxyl groups, preferably propylene glycol, sorbitol, and pentaerythritol. For example, compositions are preferred which contain, in weight percentage (USP): 18 to 45 wt. % of an activated aluminum salt, 55 to 82 wt. % of at least one anhydrous polyvalent alcohol with 3 to 6 carbon atoms and 3 to 6 hydroxyl groups, preferably 1,2-propylene glycol, 1,3-butylene glycol, diethylene glycol, dipropylene glycol, glycerol, sorbitol, and pentaerythritol, particularly preferably 1,2-propylene glycol.

Also particularly preferred are complexes of activated perspiration-inhibiting aluminum salts with a polyvalent alcohol which contain 20 to 50 wt. %, particularly preferably 20 to 42 wt. %, activated perspiration-inhibiting aluminum salt and 2 to 16 wt. % molecularly bonded water, the remainder to 100 wt. % being at least one polyvalent alcohol having 3 to 6 carbon atoms and 3 to 6 hydroxyl groups. Propylene glycol, propylene glycol/sorbitol mixtures, and propylene glycol/pentaerythritol mixtures are such preferred alcohols. Such complexes, preferred according to the present invention, of an activated perspiration-inhibiting aluminum salt with a polyvalent alcohol are disclosed, e.g. in U.S. Pat. No. 5,643,558 and U.S. Pat. No. 6,245,325.

Further preferred perspiration-inhibiting active substances are basic calcium aluminum salts as disclosed, e.g. in U.S. Pat. No. 2,571,030. These salts are produced by reacting calcium carbonate with aluminum chlorohydroxide or aluminum chloride and aluminum powder, or by adding calcium chloride dihydrate to aluminum chlorohydroxide.

Further preferred perspiration-inhibiting active substances are activated aluminum salts as disclosed, e.g. in U.S. Pat. No. 6,245,325 or U.S. Pat. No. 6,042,816, containing 5 to78 wt. % (USP) of an activated perspiration-inhibiting aluminum salt, an amino acid or hydroxyalkanoic acid in such an amount that an (amino acid or hydroxyalkanoic acid) to Al weight ratio of 2:1 to 1:20 and preferably 1:1 to 1:10 is provided, and a water-soluble calcium salt in such an amount that a Ca:(Al+Zr) weight ratio of 1:1 to 1:28 and preferably 1:2 to 1:25 is provided. Particularly preferred solid activated perspiration-inhibiting salt compositions, e.g. according to U.S. Pat. No. 6,245,325 or U.S. Pat. No. 6,042,816, contain 48 to 78 wt. % (USP), preferably 66 to 75 wt. %, of an activated aluminum salt, and 1 to 16 wt. %, preferably 4 to 13 wt. % of molecularly bonded water (hydration water), as well as water-soluble calcium salt in an amount that the Ca:Al weight ratio is 1:1 to 1:28, preferably 1:2 to 1:25, and amino acid in an amount that the amino acid to Al weight ratio is 2:1 to 1:20, preferably 1:1 to 1:10.

Further particularly preferred solid activated perspiration-inhibiting salt compositions, e.g. according to U.S. Pat. No. 6,245,325 or U.S. Pat. No. 6,042,816, contain 48 to 78 wt. % (USP), preferably 66 to 75 wt. %, of an activated aluminum salt, and 1 to 16 wt. %, preferably 4 to 13 wt. % of molecularly bonded water (hydration water), as well as water-soluble calcium salt in an amount that the Ca:Al weight ratio is 1:1 to 1:28, preferably 1:2 to 1:25, and glycine in an amount that the glycine to Al weight ratio is 2:1 to 1:20, preferably 1:1 to 1:10.

Further particularly preferred solid activated perspiration-inhibiting salt compositions, e.g. according to U.S. Pat. No. 6,245,325 or U.S. Pat. No. 6,042,816, contain 48 to 78 wt. % (USP), preferably 66 to 75 wt. %, of an activated aluminum salt, and 1 to 16 wt. %, preferably 4 to 13 wt. % of molecularly bonded water, as well as water-soluble calcium salt in an amount that the Ca:Al weight ratio is 1:1 to 1:28, preferably 1:2 to 1:25, and hydroxyalkanoic acid in an amount that the hydroxyalkanoic acid to Al weight ratio is 2:1 to 1:20, preferably 1:1 to 1:10.

Amino acids preferred for stabilizing the perspiration-inhibiting salts are selected from glycine, alanine, leucine, isoleucine, β-alanine, valine, cysteine, serine, tryptophan, phenylalanine, methionine, β-amino-n-butyric acid, and γ-amino-n-butyric acid and the salts thereof, each in d-form, l-form, and dl-form; glycine is particularly preferred.

Hydroxyalkanoic acids preferred for stabilizing the perspiration-inhibiting salts are selected from glycolic acid and lactic acid.

Further preferred activated aluminum salts are those of the general formula Al₂(OH)₆₋ₐXa, wherein X is Cl, Br, I, or NO₃, and “a” is a value of 0.3 to 5, preferably of 0.8 to 2.5, and particularly preferably 1 to 2, and so the molar ratio of Al:X is 0.9:1 to 2.1:1, as, e.g. disclosed in U.S. Pat. No. 6,074,632. In general, some hydration water is associatively bonded in these salts, typically 1 to 6 mol water per mol salt. Particularly preferred is aluminum chlorohydrate (i.e. X is Cl in the aforementioned formula), and specifically 5/6 basic aluminum chlorohydrate, wherein “a” is 1, and so the molar ratio of aluminum and chloride is 1.9:1 to 2.1:1.

Zirconium-free aluminum salts particularly preferred according to the invention have a molar metal to chloride ratio of 1.9 to 21 Zirconium-free aluminum sesquichlorohydrates particularly preferred according to the invention have a molar metal to chloride ratio from 1.5:1 to 1.8:1.

The agents according to the invention are preferably present as suspension of the undissolved antiperspirant active substance in an oil. Another preferred form of application is a sprayable water-in-oil emulsion which is preferably sprayed by means of a propellant. A further preferred form of application is a sprayable oil-in-water emulsion which is preferably sprayed as pump spray. The agents according to the invention are prepared as a product for application as aerosol, i.e. they are packaged in a pressure container, from which they are sprayed by means of a propellant. The agents according to the invention can further be sprayed as propellant-free pump spray or with a squeeze bottle.

Application is carried out with a spraying device. These spraying devices contain in a container a filling of the liquid viscous-flowable suspension- or powder-like perspiration-inhibiting agent according to the invention. The filling can be under the pressure of a propellant (pressurized gas cans, pressurized gas packages, aerosol packages), or it can be a mechanically operated pump atomizer without propellant gas (pump sprays/squeeze bottle). The containers have an extraction device, preferably in the form of valves which allow for the extraction of the content as mist, smoke, foam, powder, paste, or liquid jet. Especially cylindrical vessels made of metal (aluminum, tinplate, with a maximum volume of preferably 1000 ml), protected or shatterproof glass or plastic (with a maximum volume of preferably 220 ml), or shattering glass or plastic (with a maximum volume of preferably 50 to 400 ml), are possible as containers for the spraying devices. Cream-like, gelatinous, pasty, and liquid agents can be packaged, e.g. in pump, spray, or squeeze dispensers, particularly also in multi-chamber pump, multi-chamber spray, or multi-chamber squeeze dispensers. The packaging for the agents according to the invention can be non-transparent but also transparent or translucent.

According to the invention, the term liquid also includes any solid-state dispersions in liquids.

In a preferred embodiment, the agents according to the invention are present as water-in-oil emulsion which contains methanesulphonic acid and/or at least one physiologically compatible salt of methanesulphonic acid, preferably in a total amount of 0.05 to 5 wt. %, preferably 0.1 to 3 wt. %, particularly preferably 0.5 to 2.5 wt. %, extraordinarily preferably 1 to 2 wt. %, in each case based on the total weight of the agent without taking into account an optionally present propellant. In another preferred embodiment, the agents according to the invention are present as oil-in-water emulsion which contains methanesulphonic acid and/or at least one physiologically compatible salt of methanesulphonic acid in a total amount of 0.05 to 5 wt. %, preferably 0.1 to 3 wt. %, particularly preferably 0.5 to 2.5 wt. %, extraordinarily preferably 1 to 2 wt. %, in each case based on the total weight of the agent without taking into account an optionally present propellant.

In a further preferred embodiment, the agents according to the invention are characterized in that they are an antiperspirant water-in-oil emulsion, containing methanesulphonic acid and/or at least one physiologically compatible salt of methanesulphonic acid, preferably in a total amount of 0.05 to 5 wt. %, more preferably 0.1 to 3 wt. %, particularly preferably 0.5 to 2.5 wt. %, extraordinarily preferably 1 to 2 wt. %, further 12 to 90 wt. %, preferably 25 to 55 wt. %, particularly preferably 30 to 50 wt. %, extraordinarily preferably 35 to 45 wt. % water, at least one emulsifying agent and at least one cosmetic fat or oil, the specifications in wt. % each referring to the total weight of the agent according to the invention without taking into account an optionally present propellant.

In a further preferred embodiment, the agents according to the invention are characterized in that they are an antiperspirant oil-in-water emulsion, containing methanesulphonic acid and/or at least one physiologically compatible salt of methanesulphonic acid, preferably in a total amount of 0.05 to 5 wt. %, preferably 0.1 to 3 wt. %, particularly preferably 0.5 to 2.5 wt. %, extraordinarily preferably 1 to 2 wt. %, further 15 to 90 wt. %, preferably 25 to 55 wt. %, particularly preferably 30 to 50 wt. %, extraordinarily preferably 35 to 45 wt. % water, at least one emulsifying agent and at least one cosmetic fat or oil, the specifications in wt. % each referring to the total weight of the agent according to the invention without taking into account an optionally present propellant.

In a further preferred embodiment, the agents according to the invention are characterized in that they contain methanesulphonic acid and/or at least one physiologically compatible salt of methanesulphonic acid, preferably in a total amount of 0.05 to 5 wt. %, more preferably 0.1 to 3 wt. %, particularly preferably 0.5 to 2.5 wt. %, extraordinarily preferably 1 to 2 wt. %, and water in a total amount of 15 to 90 wt. %, preferably 25 to 70 wt. %, particularly preferably 30 to 60 wt. %, extraordinarily preferably 35 to 50 wt. %, and further ethanol in a total amount of 5 to 60 wt. %, preferably 10 to 40 wt. %, particularly preferably 15 to 35 wt. %, extraordinarily preferably 20 to 30 wt. %, and at least one hydrogel-forming substance in a total amount of 0.01 to 2 wt. %, preferably 0.1 to 1 wt. %, more preferably 0.2 to 0.7 wt. %, extraordinarily preferably 0.3 to 0.5 wt. %, the specifications in wt. % each referring to the total weight of the agent according to the invention without taking into account an optionally present propellant; particularly preferred, the agents according to the invention have a dynamic viscosity in the range of 10 to 1500 mPas, preferably 100 to 1000 mPas, particularly preferably 200 to 800 mPas, measured with a Brookfield viscosimeter, spindle RV 4, 20 s⁻¹, without Helipath, at 20° C. ambient temperature and 20° C. sample temperature.

In a further prefened embodiment, the agents according to the invention are characterized in that they contain methanesulphonic acid and/or at least one physiologically compatible salt of methanesulphonic acid, preferably in a total amount of 0.05 to 5 wt. %, preferably 0.1 to 3 wt. %, particularly preferably 0.5 to 2.5 wt. %, extraordinarily preferably 1 to 2 wt. %, and water in a total amount of 15 to 90 wt. %, preferably 25 to 70 wt. %, particularly preferably 30 to 60 wt. %, extraordinarily preferably 35 to 50 wt. %, and further ethanol in a total amount of 5 to 60 wt. %, preferably 10 to 40 wt. %, particularly preferably 15 to 35 wt. %, extraordinarily preferably 20 to 30 wt. %, and at least one hydrogel-forming substance in a total amount of 0.01 to 2 wt. %, preferably 0.1 to 1 wt. %, preferably 0.2 to 0.7 wt. %, extraordinarily preferably 0.3 to 0.5 wt. %, the specifications in wt. % each referring to the total weight of the agent according to the invention without taking into account an optionally present propellant.

In a further preferred embodiment, the agents according to the invention are characterized in that they are present as a water-in-oil emulsion containing methanesulphonic acid and/or at least one physiologically compatible salt of methanesulphonic acid, preferably in a total amount of 0.05 to 5 wt. %, preferably 0.1 to 3 wt. %, particularly preferably 0.5 to 2.5 wt. %, extraordinarily preferably 1 to 2 wt. %, and water in a total amount of 15 to 75 wt. %, preferably 25 to 60 wt. %, particularly preferably 30 to 55 wt. %, extraordinarily preferably 35 to 50 wt. %, the specifications in wt. % each referring to the total weight of the agent according to the invention without taking into account an optionally present propellant.

In another preferred embodiment, the agents according to the invention are characterized in that they are present as an oil-in-water emulsion containing methanesulphonic acid and/or at least one physiologically compatible salt of methanesulphonic acid, preferably in a total amount of 0.05 to 5 wt. %, preferably 0.1 to 3 wt. %, particularly preferably 0.5 to 2.5 wt. %, extraordinarily preferably 1 to 2 wt. %, and water in a total amount of 15 to 90 wt. %, preferably 25 to 70 wt. %, particularly preferably 30 to 60 wt. %, extraordinarily preferably 35 to 50 wt. %, and ethanol in a total amount of 5 to 50 wt. %, preferably 10 to 40 wt. %, particularly preferably 15 to 35 wt. %, extraordinarily preferably 20 to 30 wt. %, and optionally a hydrogel-forming substance in a total amount of 0.01 to 2 wt. %, preferably 0.1 to 1 wt. %, more preferably 0.2 to 0.7 wt. %, extraordinarily preferably 0.3 to 0.5 wt. %, the specifications in wt. % each referring to the total weight of the agent according to the invention without taking into account an optionally present propellant.

Hydrogel-forming substances preferred according to the invention are selected from cellulose ethers, especially hydroxyalkyl celluloses, particularly hydroxypropyl cellulose, hydroxypropyl methylcellulose, hydroxyethyl cellulose, carboxymethyl cellulose, cetyl hydroxyethyl cellulose, hydroxybutyl methyl cellulose, methyl hydroxyethyl cellulose, as well as xanthan gum, sclerotium gum, succinoglucans, polygalactomannans, particularly guar gums and locust bean gum, particularly guar gum and locust bean gum itself and the nonionic hydroxyalkyl guar derivatives and locust bean gum derivatives, such as hydroxypropyl guar, carboxymethyl hydroxypropyl guar, hydroxypropylmethyl guar, hydroxyethyl guar, and carboxymethyl guar, furthermore pectins, agar, carragheen (carrageenan), tragacanth, gum arabic, karaya gum, tara gum, gellan, gelatin, casein, propylene glycol alginate, alginic acids and the salts thereof, particularly sodium alginate, potassium alginate, and calcium alginate, furthermore polyvinylpyrrolidones, polyvinyl alcohols, polyacrylamides, furthermore—although less preferably—physically modified (e.g. by pregelatinisation) and/or chemically modified starches, particularly hydroxypropylated starch phosphates and starch octenylsuccinates and the aluminum, calcium, or sodium salts thereof, furthermore—also less preferably—acrylic acid-acrylate copolymers, acrylic acid-acrylamide copolymers, acrylic acid-vinylpyrrolidone copolymers, acrylic acid-vinyl-formamide copolymers, and polyacrylates. Particularly preferred hydrogel-forming agents are selected from cellulose ethers, principally from hydroxyalkyl celluloses, particularly from hydroxypropyl cellulose, hydroxypropylmethyl cellulose, hydroxyethyl cellulose, carboxymethyl cellulose, cetyl hydroxyethyl cellulose, hydroxybutyl methyl cellulose, and methyl hydroxyethyl cellulose, as well as mixtures thereof. Hydroxyethyl cellulose and is an extraordinarily preferred hydrogel-forming agent.

In order to further support the discoloration-inhibiting effect of methanesulphonic acid and/or the at least one physiologically compatible salt of methanesulphonic acid, preferably in a total amount of 0.05 to 5 wt. %, more preferably 0.1 to 3 wt. %, particularly preferably 0.5 to 2.5 wt. %, extraordinarily preferably 1 to 2 wt. %, it can be advantageous to add to the agents according to the invention at least one chelating agent that is selected from ethylenediaminetetraacetic acid (EDTA) and the salts thereof, and from nitrilotriacetic acid (NTA) and mixtures of these substances, in a total amount from 0.01 to 0.5 wt. %, preferably 0.02 to 0.3 wt. %, particularly preferably 0.05 to 0.1 wt. %.

Further agents preferred according to the invention are therefore characterized in that additionally at least one chelating agent selected from ethylenediaminetetraacetic acid (EDTA) and the salts thereof, and from nitrilotriacetic acid (NTA) and mixtures of these substances, is contained in a total amount of 0.01 to 0.5 wt. %, preferably 0.02 to 0.3 wt. %, particularly preferably 0.05 to 0.1 wt. %, the specifications in wt. % each referring to the total weight of the agent without taking into account an optionally present propellant.

Further compositions preferred according to the invention optionally contain at least one deodorant active substance in a total amount of 0.0001 to 40 wt. %, preferably 0.2 to 20 wt. %, particularly preferably 1 to 15 wt. %, extraordinarily preferably 1.5 to 5 wt. %, the specifications in wt. % referring to the total weight of the composition without taking into account an optionally present propellant.

According to the invention, ethanol is not regarded as a deodorant active substance but instead, if present, merely as a component of the carrier.

In a preferred embodiment, the agents according to the invention contain as a deodorizing active substance at least one silver salt preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate, and silver galactarate, and from mixtures of these salts. Silver sulfate, silver citrate, silver dihydrogen citrate, and silver lactate, as well as mixtures of these salts, are extraordinarily preferred.

Further preferred compositions according to the invention contain at least one silver salt preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate, and silver galactarate, and from mixtures of these salts, in such amounts that silver is contained in a total amount of 1 to 100 ppm, preferably 2 to 50 ppm, particularly preferably 5 to 20 ppm, extraordinarily preferably 7 to 10 ppm, in each case based on the weight of the propellant-free composition. The correspondingly required amount of silver salt(s) can be calculated based on the molar masses of silver (107.87 g/mol) and of the corresponding silver salts—silver lactate, e.g., has a molar mass of 196.94 g/mol.

In a further preferred embodiment, the agents according to the invention contain as a deodorizing active substance at least one aromatic alcohol of structure (AA-1)

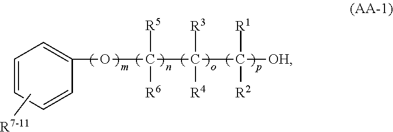

wherein
the groups R¹ to R⁶, independently from one another, denote a hydrogen atom, an alkyl group having 1 to 10 carbon atoms, which can be linear or branched and substituted with OH groups or alkoxy groups having 1 to 5 carbon atoms, or an alkenyl group having 2 to 10 carbon atoms, which can be linear or branched and substituted with OH groups or alkoxy groups having 1 to 5 carbon atoms,
the groups R⁷ to R¹¹, independently from one another, denote a hydrogen atom, a halogen atom, particularly a chlorine atom, or an alkyl group having 1 to 10 carbon atoms, which can be linear or branched and substituted with OH groups or alkoxy groups having 1 to 5 carbon atoms, particularly with a methoxy group,
m=0 or 1, n, o, p=independently from one another integers from 0 to 10, wherein at least one of the values n, o, p≠0.

Particularly preferred products according to the invention contain at least one alcohol AA-1 as described above, which is selected from anise alcohol, 2-methyl-5-phenyl-pentan-1-ol, 1,1-dimethyl-3-phenyl-propan-1-ol, benzyl alcohol, 2-phenylethan-1-ol, 3-phenylpropan-1-ol, 4-phenylbutan-1-ol, 5-phenylpentan-1-ol, 2 benzylheptan-1-ol, 2,2-dimethyl-3-phenylpropan-1-ol, 2,2 dimethyl-3-(3′-methylphenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-ol, 2-ethyl-3-(2′-methylphenyl)-propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3′,4′-dimethoxyphenyl)-2-ethylpropan-1-ol, 2-allyl-3-phenylpropan-1-ol, and 2 n-pentyl-3-phenylpropan-1-ol, as well as mixtures thereof 2-Benzylheptan-1-ol, as well as mixtures of 2-benzylheptan-1-ol and phenoxyethanol, are extraordinarily preferred. Further particularly preferred compositions according to the invention contain at least one alcohol AA-1, as described above, in a total amount from 0.05 to 10 wt. %, preferably 0.1 to 5 wt. %, particularly preferably 0.2 to 2 wt. %, extraordinarily preferably 0.3 to 1.5 wt. %, in each case based on the weight of the composition without taking into account an optionally present propellant. Extraordinarily preferred agents according to the invention contain 2-benzylheptan-1-ol in a total amount of 0.05 to 1.5 wt. %, preferably 0.1 to 1 wt. %, particularly preferably 0.2 to 0.5 wt. %, in each case based on the weight of the composition without taking into account an optionally present propellant.

In a further preferred embodiment, the agents according to the invention contain as a deodorant active substance at least one 1,2-alkanediol having 5 to 12 carbon atoms, which is described by the formula HO—CH₂—CH(OH)—(CH2)ₙ-CH₃, in which n denotes the numbers 2, 3, 4, 5, 6, 7, 8, or 9, as well as mixtures of these 1,2-alkanediols. 1,2-Alkanediols having 5 to 12 carbon atoms that are particularly preferred according to the invention are selected from 1,2-pentanediol, 1,2-hexanediol, 1,2-octanediol, 1,2-decanediol, and mixtures thereof. A very particularly preferred combination according to the invention are mixtures of 1,2-hexanediol and 1,2-octanediol, preferably at a weight ratio of 10:1 to 1:10, more preferably of 5:1 to 1:5, particularly preferably at a weight ratio of 1:1.

Preferred agents according to the invention contain at least one 1,2-alkanediol having 5 to 12 carbon atoms, which is described by the formula HO—CH₂—CH(OH)—(CH2)ₙ-CH₃, in which n denotes the numbers 2, 3, 4, 5, 6, 7, 8, or 9, in a total amount of 0.2 to 15 wt. %, preferably 0.3 to 10 wt. %, particularly preferably 0.4 to 5 wt. %, and extraordinarily preferably 0.5 to 2 wt. %, in each case based on the weight of the composition without taking into account an optionally present propellant. Extraordinarily preferred agents according to the invention contain 0.2 to 0.5 wt. % 1,2-hexanediol and 0.2 to 0.5 wt. % 1,2-octanediol, in each case based on the weight of the composition without taking into account an optionally present propellant.

Further preferred agents according to the invention are characterized by containing the deodorant active substance 3-(2-ethylhexyloxy)-1,2-propanediol, preferably in a total amount from 0.05 to 5 wt. %, more preferably 0.1 to 2 wt. %, particularly preferably 0.2 to 1.5 wt. %, extraordinarily preferably 0.5 to 1.0 wt. %, in each case based on the weight of the composition without taking into account an optionally present propellant.

Further preferred agents according to the invention are characterized by containing tropolone (2-hydroxy-2,4,6-cycloheptatrienone), preferably in an amount of 0.001 to 0.1 wt. %, based on the weight of the composition without taking into account an optionally present propellant.

Further preferred agents according to the invention are characterized by containing the deodorant active substance triethyl citrate. Triethyl citrate is a known deodorant active substance that acts as an enzyme inhibitor for esterases and lipases and thus contributes to the broad-spectrum effect of agents according to the invention. Preferred agents according to the invention contain 0.5 to 15 wt. %, preferably 3 to 8 wt. %, extraordinarily preferably 4 to 6 wt. %, in each case based on the weight of the composition without taking into account an optionally present propellant.

Apocrine perspiration represents a complex mixture that contains, inter alia, sebum and other fats as well as steroids. Steroids themselves are not water-soluble. In order for them to be able to be transported away with the bodily fluids, they are normally present as sulfate or glucuronide. On the skin, these steroid esters are cleaved to yield the volatile free steroids by hydrolytic enzymes of the skin bacteria, particularly of the coryneform bacteria. In principle, all bacterial exoesterases are capable of this, but particularly the enzymes arylsulfatase and beta-glucuronidase. Compounds that inhibit aryl sulfatase or beta-glucuronidase therefore represent deodorant active substances preferred according to the invention.

Development of the short- and medium-chain fatty acids that contribute substantially to body odor begins with the cleavage of skin lipids to yield branched long-chain fatty acids. Cleavage of the skin lipids, which are present predominantly as glycerol esters, is accomplished substantially by propionibacterium, corynebacterium A, and staphylococcus species (A. G. James et al., Generation and Turnover of Volatile Fatty Acids by Axillary Bacteria, 22^{nd} IFSCC Congress, Edinburgh, 2002, Poster 108). A. G. James et al. further disclose that both short-chain C₂ to C₅ fatty acids and medium-chain C₆ to C₁₂ fatty acids, which are principally responsible for axillary body odor, are formed from the long-chain branched fatty acids by the hydrolytic enzymes of a specific corynebacterium that A. G. James et al. refer to as corynebacterium A. In principle, all bacterial exoesterases are capable of this lipid cleavage, but particularly the enzyme lipase. Compounds that inhibit lipase therefore likewise represent deodorant active substances preferred according to the invention.

A further class of compounds that likewise forms during bacterial decomposition of perspiration components and contributes to body odor are saturated and unsaturated aldehydes, primarily those having a chain length of C₆ to C₁₂, particularly hexanal, heptanal, octenal, and nonenal. These are produced by beta-cleavage from the hydroperoxides that are formed under the effect of 5-lipoxygenase on unsaturated fatty acids. Compounds that inhibit the enzyme 5-lipoxygenase therefore likewise represent deodorant active substances preferred according to the invention.

It is furthermore known that highly foul-smelling components of human body odor and mouth odor represent so-called volatile sulfur compounds (VSCs) that are released particularly by enzymatic reaction. Sulfur-containing compounds come into contact with the human skin as water-soluble amino acid conjugates together with perspiration. There they are released by skin bacteria (chiefly staphylococci and corynebacteria) through enzymatic reaction. An enzyme that plays a particular role in the release of VSCs is cystathionine beta-lyase. This enzyme cleaves VSCs from the amino acids and is thus an important cause in the development of body odor. Compounds that inhibit the enzyme cystathionine beta-lyase therefore likewise represent deodorant active substances preferred according to the invention.

Further preferred agents according to the invention are characterized by containing at least one compound that is an inhibitor of the enzyme arylsulfatase. Deodorant active substances preferred according to the invention that act as an arylsulfatase inhibitor are those disclosed, for example, in U.S. Pat. No. 5,643,559, U.S. Pat. No. 5,676,937, WO2001/099376 A2, EP 1430879 A1, and DE 10216368 A1. Further particularly preferred agents according to the invention are characterized by containing at least one compound that is an inhibitor of the enzyme arylsulfatase in a total amount of 0.001 to 10 wt. %, preferably 0.01 to 5 wt. %, particularly preferably 0.1 to 2.5 wt. %, in each case based on the weight of the composition without taking into account an optionally present propellant.

Further preferred agents according to the invention are characterized by containing at least one compound that is an inhibitor of the enzyme beta-glucuronidase. Deodorant active substances preferred according to the invention that act as beta-glucuronidase inhibitors are those disclosed, for example, in WO2003/039505 A2. Further particularly preferred agents according to the invention are characterized by containing at least one compound that is an inhibitor of the enzyme beta-glucuronidase in a total amount of 0.001 to 10 wt. %, preferably 0.01 to 5 wt. %, particularly preferably 0.1 to 2.5 wt. %, in each case based on the weight of the composition without taking into account an optionally present propellant.

Further preferred agents according to the invention are characterized by containing at least one compound that is an inhibitor of the enzyme lipase. Deodorant active substances preferred according to the invention that act as a lipase inhibitor are selected from those disclosed in EP 1428520 A2, furthermore selected from the aminomethylenemalonic acid derivatives according to DE 3018132 A1, the ethylene oxide-propylene oxide copolymers according to GB 2335596 A1, and the salts of phytic acid according to EP 650 720 A1. Further particularly preferred agents according to the invention are characterized by containing at least one compound that is an inhibitor of the enzyme lipase in a total amount of 0.001 to 10 wt %, preferably 0.01 to 5 wt. %, particularly preferably 0.1 to 2.5 wt. %, in each case based on the weight of the composition without taking into account an optionally present propellant.

Further particularly preferred agents according to the invention are characterized by containing at least one compound that is an inhibitor of 5-lipoxygenase. Deodorant active substances preferred according to the invention that act as a 5-lipoxygenase inhibitor are disclosed in EP 1428519 A2. Further particularly preferred agents according to the invention are characterized by containing at least one compound that is an inhibitor of the enzyme 5-lipoxygenase in a total amount of 0.001 to 10 wt %, preferably 0.01 to 5 wt. %, particularly preferably 0.1 to 2.5 wt. %, in each case based on the weight of the composition without taking into account an optionally present propellant.

Further particularly preferred agents according to the invention are characterized by containing at least one compound that is an inhibitor of the enzyme cystathionine beta-lyase. Deodorant active substances preferred according to the invention that act as an inhibitor of cystathionine beta-lyase are selected from those disclosed in EP 495918 B1, WO 2006/079934, DE 102010000746 A1, WO2010/031657 A1, and WO2010/046291 A1. Further particular preferred agents according to the invention are characterized by containing at least one compound that is an inhibitor of the enzyme cystathionine beta-lyase in a total amount of 0.001 to 10 wt %, preferably 0.01 to 5 wt. %, particularly preferably 0.1 to 2.5 wt. %, in each case based on the weight of the composition without taking into account an optionally present propellant.

Further particularly preferred agents according to the invention can be characterized as including at least one cationic phospholipid of formula KPL

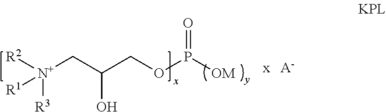

in which R¹ is an alkyl, alkenyl, or hydroxyalkyl group having 8 to 22 carbon atoms or an acylaminoalkyl group of the formula R⁵CONH(CₘH₂ₘ)—, in which R⁵CO is a linear acyl group having 8 to 22 carbon atoms and m=2 or 3, R² and R³ are alkyl groups having 1 to 4 carbon atoms or hydroxyalkyl groups having 2 to 4 carbon atoms or carboxyalkyl groups of the formula —(CH₂)_{z}—COOM, in which z has a value from 1 to 3 and M is hydrogen or an alkali metal cation, x has a value from 1 to 3, and y a value of (3-x), M is hydrogen or an alkali metal cation, and A⁻ is an anion.

Preferred alkyl groups having 8 to 22 carbon atoms are selected from an n-octyl, n-nonyl, n-decyl, n-undecyl, lauryl, n-tridecanyl, myristyl, n-pentadecanyl, cetyl, palmityl, stearyl, elaidyl, arachidyl, behenyl, and a cocyl group. A representative cocyl group consists, based on its total weight, of 4 to 9 wt. % n-octyl groups, 4 to 9 wt. % n-decyl groups, 45 to 55 wt. % lauryl groups, 15 to 21 wt. % myristyl groups, 8 to 13 wt. % palmityl groups, and 7 to 14 wt. % stearyl groups. Preferred alkenyl groups having 8 to 22 carbon atoms are selected from a linoleyl group ((9Z,12Z)-octadeca-9,12-dien-1-yl) and a linolenyl group ((9Z,12Z,15Z)-octadeca-9,12,15-trien-1-yl). A preferred hydroxyalkyl group having 8 to 22 carbon atoms is selected from a 12-hydroxystearyl group.

Particularly preferred cationic phospholipids of formula IPL are those in which R¹ is an acylaminoalkyl group of the formula R⁵CONH(CₘH₂ₘ)—, in which R⁵CO represents a linear acyl group having 8 to 22 carbon atoms and m=3.

Preferred linear acyl groups R⁵CO having 8 to 22 carbon atoms are selected from an n-octanoyl, n-nonanoyl, n-decanoyl, n-undecanoyl, lauroyl, n-tridecanoyl, myristoyl, n-pentadecanoyl, cetoyl, palmitoyl, stearoyl, elaidoyl, arachidoyl, behenoyl, and a cocoyl group. A representative cocoyl group consists, based on its total weight, of 4 to 9 wt. % n-octanoyl groups, 4 to 9 wt. % n-decanoyl groups, 45 to 55 wt. % lauroyl groups, 15-21 wt. % myristoyl groups, 8 to 13 wt. % palmitoyl groups, and 7 to 14 wt. % stearoyl groups. Particularly preferred linear acyl groups R⁵CO are selected from a cocoyl group, a lauroyl group (n-C₁₁H₂₃CO), a myristoyl group (n-C₁₃H₂₇CO), and a linoleoyl group ((9Z,12Z)-octadeca-9,12-dien-1-oyl). Extraordinarily preferred linear acyl groups R⁵CO are selected from a cocoyl group, a lauroyl group (n-C₁₁H₂₃CO), and a myristoyl group (n-C₁₃H₂₇CO).

Preferred alkyl groups having 1 to 4 carbon atoms are a methyl, ethyl, n-propyl, 1-methylethyl, n-butyl, 2-methylpropyl, and tert-butyl group. The methyl group is particularly preferred.

Preferred hydroxyalkyl groups having 2 to 4 carbon atoms are a 2-hydroxyethyl group and a 1-hydroxyethyl group.

Preferred carboxyalkyl groups of the formula —(CH₂)_{z}—COOM, in which z=1 to 3, are a carboxymethyl, a carboxyethyl, and a carboxy-n-propyl group.

Preferred alkali metal cations are selected from sodium and potassium cations; Na⁺ is particularly preferred. Preferred anions are selected from sulfate, chloride, phosphate, nitrate, hydrogen carbonate, and acetate, a chloride anion being particularly preferred.

Preferred agents according to the invention can include, as a deodorizing active substance, a cationic phospholipid of formula KPL

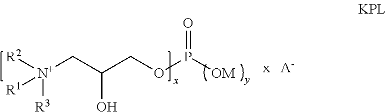

in which R¹ is an acylaminoalkyl group of the formula R⁵CONH(CₘH₂ₘ)—, in which R⁵CO is selected from a cocoyl group, a lauroyl group, a myristoyl group, and a linoleoyl group, and m=3, R² and R³ are methyl groups, x=2, y=1, M is a sodium ion, and A⁻ is a chloride ion. Preferably at least one cationic phospholipid of formula KPL having the features mentioned above is contained in a total amount of 0.05 to 2 wt. %, preferably 0.1 to 1 wt. %, particularly preferably 0.15 to 0.4 wt. %, in each case based on the weight of the agent without taking into account an optionally present propellant.

Particularly preferred agents according to the invention can include a cationic phospholipid of formula KPL

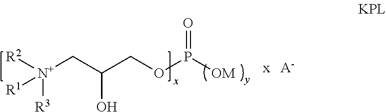

in which R¹ is a cocoylaminopropyl group (also referred to as a cocamidopropyl group), R² and R³ are methyl groups, x=2, y=1, M is a sodium ion, and A⁻ is a chloride ion, and which is obtainable under the INCI name Cocoamidopropyl PG-Dimonium Chloride Phosphate, in a total amount of 0.05 to 2 wt. %, preferably 0.1 to 1 wt. %, particularly preferably 0.15 to 0.4 wt. %, in each case based on the weight of the agent without taking into account an optionally present propellant.

Further, particularly preferred agents according to the invention can include a cationic phospholipid of formula KPL

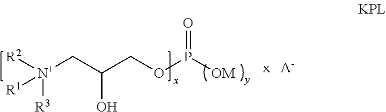

in which R¹ is a myristoylaminopropyl group, R² and R³ are methyl groups, x=2, y=1, M is a sodium ion, and A⁻ is a chloride ion, and which is obtainable under the INCI name Myristoamidopropyl PG-Dimonium Chloride Phosphate, in a total amount of 0.05 to 2 wt. %, preferably 0.1 to 1 wt. %, particularly preferably 0.15 to 0.4 wt. %, in each case based on the weight of the agent without taking into account an optionally present propellant.

Further, particularly preferred agents according to the invention can include a cationic phospholipid of formula KPL

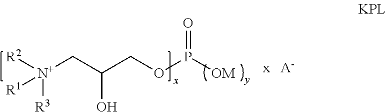

in which R¹ is a lauroylaminopropyl group, R² and R3 are methyl groups, x=2, y=1, M is a sodium ion, and A⁻ is a chloride ion, in a total amount of 0.05 to 2 wt. %, preferably 0.1 to 1 wt. %, particularly preferably 0.15 to 0.4 wt. %, in each case based on the weight of the agent without taking into account an optionally present propellant.

Further deodorant active substances preferred according to the invention are odor absorbers, ion exchangers having a deodorizing effect, germ-inhibiting agents, prebiotically effective components, and enzyme inhibitors, or, particularly preferably, combinations of the aforementioned active substances.

Silicates serve as odor absorbers which simultaneously also advantageously assist the rheological properties of the composition according to the invention. Included in the silicates particularly preferred according to the invention are mainly sheet silicates, and among those particularly montmorillonite, kaolinite, illite, beidellite, nontronite, saponite, hectorite, bentonite, smectite, and talc. Further preferred odor absorbers are, for example, zeolites, zinc ricinoleate, cyclodextrins, specific metal oxides such as e.g. aluminum oxide, and chlorophyll. They are contained preferably in a total amount of 0.1 to 10 wt. %, particularly preferably 0.5 to 7 wt. %, and extraordinarily preferably 1 to 5 wt. %, in each case based on the total composition without propellant.

Germ-inhibiting or antimicrobial active substances according to the invention are those active substances which reduce the number of skin germs participating in odor formation, and/or inhibit their growth. Included among these germs are, among others, various species from the group of staphylococci, the group of corynebacteria, anaerococci, and micrococci.

Germ-inhibiting or antimicrobial active substances preferred according to the invention are particularly organohalogen compounds and organohalogen halides, quaternary ammonium compounds, a number of plant extracts, and zinc compounds. These include, among others, triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphen bromide, ammonium phenolsulfonate, benzalkonium halides, benzalkonium cetyl phosphate, benzalkonium saccharinate, benzethonium chloride, cetylpyridinium chloride, laurylpyridinium chloride, laurylisoquinolinium bromide, methylbenzethonium chloride. In addition, phenol, phenoxyethanol, disodiumdihydroxyethylsulfosuccinyl undecylenate, sodium bicarbonate, zinc lactate, sodium phenolsulfonate and zinc phenolsulfonate, ketoglutaric acid, terpene alcohols such as e.g. farnesol, chlorophyllin-copper complexes, α-monoalkylglycerol ethers with a branched or linear, saturated or unsaturated, optionally hydroxylated C₆ to C₂₂ alkyl group, particularly preferably α-(2-ethylhexyl)glycerol ether, available commercially as Sensiva® SC 50 (ex Schülke & Mayr), as well as carboxylic acid esters of mono-, di-, and triglycerol (e.g. glycerol monolaurate, diglycerol monocaprinate) are preferred deodorant active substances.

Preferred antiperspirant agents according to the invention contain at least one deodorant active substance that is selected from silver salts, aromatic alcohols of structure AA-1 having the substituents mentioned above, 1,2-alkanediols having 5 to 12 carbon atoms, alpha-(2-ethylhexyl)glycerol ether (3-(2-ethylhexyloxy)-1,2-propanediol), tropolone, triethyl citrate, cationic phospholipids of formula KPL having the aforementioned substituents, as well as mixtures thereof.

Further preferred antiperspirant compositions according to the invention are characterized in that the at least one deodorant active substance is contained in a total amount of 0.0001 to 20 wt. %, preferably 1 to 15 wt. %, particularly preferably 1.5 to 5 wt. %, the specifications in wt. % referring to the total weight of the composition without taking into account an optionally present propellant.

In a further particularly preferred embodiment, the compositions according to the invention contain both at least one deodorant active substance in a total amount of 0.0001 to 40 wt. %, preferably 0.2 to 20 wt. %, particularly preferably 1 to 15 wt. %, extraordinarily preferably 1.5 to 5 wt. %, and at least one antiperspirant aluminum salt in a total amount of 2 to 40 wt. %, preferably 8 to 35 wt. %, particularly preferably 10 to 28 wt. %, and extraordinarily preferably 12 to 20 wt. %, the specifications in wt. % referring to the total weight of the active substance (USP), free of water of crystallization and free of ligands, in the composition without taking into account an optionally present propellant.

Preferred agents according to the invention preferably further contain at least one cosmetic oil that is liquid under noiival conditions and is neither an odorous substance nor an essential oil.

The cosmetic oil is liquid under normal conditions. According to the invention, essential oils are considered to be mixtures of volatile components that are produced by steam distillation from vegetable raw materials, e.g. citrus oils.

In compositions preferred according to the invention, the total amount of cosmetic oils that are liquid under normal conditions and are not an odorous substance and not an essential oil is 1 to 95 wt. %, preferably 5 to 90 wt. %, particularly preferably 30 to 75 wt. %, extraordinarily preferably 50 to 60 wt. %, the amounts referring to the weight of the composition without taking into account an optionally present propellant.

Further agents preferred according to the invention contain at least one cosmetic oil that is not an odorous substance and not an essential oil in a total amount of 1 to 70 wt. %, preferably 5 to 50 wt. %, particularly preferably 9 to 25 wt. %, extraordinarily preferably 15 to 20 wt. %, in each case based on their total weight without taking into account an optionally present propellant.

A distinction is made in the context of cosmetic oils between volatile and nonvolatile oils. Nonvolatile oils are those oils that, at 20° C. and an ambient pressure of 1013 hPa, have a vapor pressure of less than 2.66 Pa (0.02 mm Hg). Volatile oils are those oils that, at 20° C. and an ambient pressure of 1013 hPa, have a vapor pressure of 2.66 Pa to 40,000 Pa (0.02 mm to 300 mm Hg), preferably 10 to 12,000 Pa (0.1 to 90 mm Hg), particularly preferably 13 to 3000 Pa, extraordinarily preferably 15 to 500 Pa.

Volatile cosmetic oils are usually selected from among cyclic silicone oils having the INCI name Cyclomethicones. The INCI name Cyclomethicone is understood particularly to mean cyclotrisiloxane (hexamethylcyclotrisiloxane), cyclotetrasiloxane (octamethylcyclotetrasiloxane), cyclopentasiloxane (decamethylcyclopentasiloxane), and cyclohexasiloxane (dodecamethylcyclohexasiloxane). These oils have a vapor pressure of approximately 13 to 15 Pa at 20° C.

Cyclomethicones are known in the prior art as oils well suited for cosmetic compositions, particularly for deodorizing compositions such as sprays and sticks. Due to their persistence in the environment, however, it can be preferred according to the invention to omit the use of cyclomethicones. In an especially preferred embodiment, the compositions according to the invention and used according to the invention contain 0 to less than 1 wt. %, preferably a maximum of 0.1 wt. %, cyclomethicones, based on the weight of the composition without taking into account an optionally present propellant.

A cyclomethicone substitute preferred according to the invention is a mixture of C₁₃ to C₁₆ isoparaffins, C₁₂ to C₁₄ isoparaffins, and C13 to C15 alkanes, the viscosity of which at 25° C. is in the range of 2 to 6 mPas and which has a vapor pressure at 20° C. in the range of 10 to 150 Pa, preferably 100 to 150 Pa. A mixture of this kind is obtainable, for example, from Presperse Inc. under the name SiClone SR-5.

Further preferred volatile silicone oils are selected from volatile linear silicone oils, particularly volatile linear silicone oils having 2 to 10 siloxane units, such as hexamethyldisiloxane (L₂), octamethyltrisiloxane (L₃), decamethyltetrasiloxane (L₄), as contained e.g. in the commercial products DC 2-1184, Dow Corning® 200 (0.65 cSt), and Dow Corning® 200 (1.5 cSt) of Dow Corning, and low-molecular-weight phenyl trimethicones having a vapor pressure at 20° C. of approximately 2000 Pa, as obtainable e.g. from GE Bayer Silicones/Momentive under the name Baysilone Fluid PD 5.

Preferred antiperspirant compositions according to the invention, due to the drier skin feel and faster release of active substances, contain at least one volatile silicone oil which can be cyclic or linear.

Further preferred compositions according to the invention, due to the drier skin feel and faster release of the antiperspirant active substance, contain at least one volatile non-silicone oil. Preferred volatile non-silicone oils are selected from C₈ to C₁₆ isoparaffins, particularly from isononane, isodecane, isoundecane, isododecane, isotridecane, isotetradecane, isopentadecane, and isohexadecane, as well as mixtures thereof. C₁₀ to C₁₃ isoparaffin mixtures are preferred, particularly those having a vapor pressure at 20° C. of 10 to 400 Pa, preferably 13 to 100 Pa.

This at least one C₈ to C₁₆ isoparaffm is contained preferably in a total amount to 1 to 60 wt. %, preferably 3 to 45 wt. %, particularly preferably 5 to 40 wt. %, extraordinarily preferably 8 to 20 wt. %, in each case based on the total weight of the composition without taking into account an optionally present propellant.

Further compositions preferred according to the invention contain at least one nonvolatile cosmetic oil selected from nonvolatile silicone oils and nonvolatile non-silicone oils. Residues of components insoluble in the composition, such as talc, but also the antiperspirant active substances (=perspiration-inhibiting aluminum salts) dried onto the skin, can be successfully masked with a nonvolatile oil. In addition, using a mixture of various oils, particularly of nonvolatile and volatile oil, parameters such as skin feel, visibility of the residue and stability of the composition according to the invention can be precisely regulated and better adapted to consumers' needs.

Agents preferred according to the invention are characterized in that the cosmetic oil that is not an odorous substance and not an essential oil comprises at least one volatile oil having a vapor pressure of 10 to 3000 Pa at 20° C. that is not an odorous substance and not an essential oil, in a total amount of 10 to 100 wt. %, particularly preferably 10 to 80 wt. %, in each case based on the total weight of the cosmetic oils.

It is of course likewise possible to formulate agents according to the invention having a small portion of volatile oils—i.e. having 0.5 to 15 wt. % volatile oils, based on the total weight of the agent—or even having no volatile oils.

Oils particularly preferred according to the invention are esters of linear or branched, saturated or unsaturated fatty alcohols having 2 to 30 carbon atoms with linear or branched, saturated or unsaturated fatty acids having 2 to 30 carbon atoms, which can be hydroxylated. It should be noted in this regard that some esters of linear or branched C₁ to C₂₂ alkanols or C₁₄ to C₂₂ alkenols, and some triesters of glycerol with linear or branched C₂ to C₂₂ carboxylic acids, which can be saturated or unsaturated, are solid under normal conditions, such as e.g. cetyl stearate or glycerol tristearate (=stearin). These esters that are solid under normal conditions do not represent cosmetic oils according to the invention, since they do not meet the criterion of “liquid under normal conditions.” The categorization as to whether such an ester is liquid or solid under normal conditions is within the common general knowledge of a person skilled in the art.

Esters of linear or branched saturated fatty alcohols having 2 to 5 carbon atoms with linear or branched, saturated or unsaturated fatty acids having 3 to 18 carbon atoms, which can be hydroxylated, are preferred. Preferred examples thereof are isopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate, and 2-ethylhexyl stearate. Also preferred are isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate, ethylene glycol dipalmitate, n-hexyl laurate, n-decyl oleate, oleyl oleate, oleyl erucate, erucyl oleate, C₁₂ to C₁₅ alkyl lactate, and di-C₁₂ to C₁₃-alkyl malate, as well as benzoic acid esters of linear or branched C₈₋₂₂ alkanols. Benzoic acid-C₁₂ to C₁₅-alkyl esters are particularly preferred, obtainable e.g. as the commercial product Finsoly® TN (C₁₂ to C₁₅ alkyl benzoate), as well as benzoic acid isostearyl esters, obtainable e.g. as Finsolv® SB, 2-ethylhexyl benzoate, obtainable e.g. as Finsolv® EB, and benzoic acid-2-octyldodecyl esters, obtainable e.g. as Finsolv® BOD.

Further oil components preferred according to the invention are selected from C₈ to C₂₂ fatty alcohol esters of monovalent or polyvalent C₂ to C₇ hydroxycarboxylic acids, particularly the esters of glycolic acid, lactic acid, malic acid, tartaric acid, citric acid, and salicylic acid. Such esters based on linear C_{14/15} alkanols, e.g. C₁₂ to C₁₅ alkyl lactate, and on C_{12/13} alkanols branched in the 2-position, can be obtained under the trade name Cosmacol® from Nordmann, Rassmann GmbH & Co., Hamburg, particularly the commercial products Cosmacol® ESI, Cosmacol® EMI, and Cosmacol® ETI.

The use of isopropyl esters of C₁₂ to C₁₈ carboxylic acids, particularly the use of isopropyl myristate, and particularly preferably mixtures of isopropyl myristate with C₁₀ to C₁₃ isoparaffin mixtures, the latter preferably having a vapor pressure of 10 to 400 Pa at 20° C., has proven particularly advantageous, for example in terms of active substance release.

Agents preferred according to the invention contain at least one ester of linear or branched, saturated or unsaturated fatty alcohols having 2 to 30 carbon atoms with linear or branched, saturated or unsaturated fatty acids having 2 to 30 carbon atoms, which can be hydroxylated, in a total amount of 1 to 30 wt. %, preferably 5 to 26 wt. %, particularly preferably 9 to 24 wt. %, extraordinarily preferably 12 to 17 wt. %, in each case based on the weight of the total composition without taking into account an optionally present propellant.

A further particularly preferred ester oil is triethyl citrate. Further products preferred according to the invention contain triethyl citrate and at least one C₈ to C₁₆ isoparaffin selected from isononane, isodecane, isoundecane, isododecane, isotridecane, isotetradecane, isopentadecane, and isohexadecane, as well as mixtures of these isoparaffins. Further products preferred according to the invention contain triethyl citrate and at least one C₈ to C₁₆ isoparaffin selected from isononane, isodecane, isoundecane, isododecane, isotridecane, as well as mixtures of these C₈ to C₁₆ isoparaffins. Further products preferred according to the invention contain triethyl citrate and a mixture of isodecane, isoundecane, isododecane, and isotridecane. Further products preferred according to the invention contain a mixture of triethyl citrate and cyclomethicone.

Further nonvolatile non-silicone oils preferred according to the invention are selected from branched, saturated or unsaturated fatty alcohols having 6 to 30 carbon atoms. These alcohols are often also referred to as Guerbet alcohols, since they are obtainable according to the Guerbet reaction. Preferred alcohol oils are 2-hexyldecanol, 2-octyldodecanol, and 2-ethylhexyl alcohol. Isostearyl alcohol is likewise preferred. Further preferred nonvolatile oils are selected from mixtures of Guerbet alcohols and Guerbet alcohol esters, e.g. 2-hexyldecanol and 2-hexyldecyl laurate.

The term “triglyceride” used in the following means “glycerol triester.” Further nonvolatile oils preferred according to the invention are selected from triglycerides of linear or branched, saturated or unsaturated, optionally hydroxylated C₈₋₃₀ fatty acids, provided they are liquid under normal conditions. The use of natural oils, e.g. soy oil, cottonseed oil, sunflower oil, palm oil, palm kernel oil, linseed oil, almond oil, castor oil, corn oil, rapeseed oil, olive oil, sesame oil, thistle oil, wheat germ oil, peach kernel oil, and the liquid components of coconut oil and the like, can be particularly suitable. Synthetic triglyceride oils are particularly preferred, particularly capric/caprylic triglycerides, e.g. the commercial products Myritol® 318 or Myritol® 331 (BASF/BASF) having unbranched fatty acid groups, as well as glyceryl triisostearin and glyceryl tri(2-ethylhexanoate) having branched fatty acid groups. Such triglyceride oils preferably account for a portion of less than 50 wt. % of the total weight of all cosmetic oils in the composition according to the invention. Particularly preferably, the total weight of triglyceride oils is 0.5 to 25 wt. %, preferably 1 to 5 wt. %, in each case based on the total composition without taking into account an optionally present propellant.

Further nonvolatile non-silicone oils particularly preferred according to the invention are selected from dicarboxylic acid esters of linear or branched C₂ to C₁₀ alkanols, particularly diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further nonvolatile non-silicone oils particularly preferred according to the invention are selected from addition products of 1 to 5 propylene oxide units to mono- or polyvalent C₈₋₂₂ alkanols, such as octanol, decanol, decanediol, lauryl alcohol, myristyl alcohol, and stearyl alcohol, e.g. PPG-2 Myristyl Ether and PPG-3 Myristyl Ether.

Further nonvolatile non-silicone oils particularly preferred according to the invention are selected from addition products of at least 6 ethylene oxide and/or propylene oxide units to mono- or polyvalent C₃₋₂₂ alkanols, such as glycerol, butanol, butanediol, myristyl alcohol, and stearyl alcohol, which can be esterified if desired, e.g. PPG-14 Butyl Ether, PPG-9 Butyl Ether, PPG-10 Butanediol, and PPG-15 Stearyl Ether.

Further nonvolatile non-silicone oils particularly preferred according to the invention are selected from symmetrical, asymmetrical, or cyclic esters of carbonic acid with C₆ to C₂₀ alcohols, e.g. di-n-caprylyl carbonate (Cetiol® CC), or di-(2-ethylhexyl) carbonate (Tegosoft DEC). Esters of carbonic acid with C₁ to C₅ alcohols, however, e.g. glycerol carbonate or propylene carbonate, are not compounds suitable as a cosmetic oil.

Further oils that can be preferred according to the invention are selected from esters of dimers of unsaturated C₁₂ to C₂₂ fatty acids (dimer fatty acids) with monovalent linear, branched, or cyclic C₂ to C₁₈ alkanols or with polyvalent linear or branched C₂ to C₆ alkanols. The total weight of dimer fatty acid esters is particularly preferably 0.5 to 10 wt. %, preferably 1 to 5 wt. %, in each case based on the total composition without taking into account an optionally present propellant.

Further cosmetic oils that are particularly preferred according to the invention are selected from nonvolatile silicone oils. Nonvolatile silicone oils preferred according to the invention are selected from linear polyalkylsiloxanes having a kinematic viscosity at 25° C. of at least 5 cSt to 2000 cSt, selected particularly from linear polydimethylsiloxanes having a kinematic viscosity at 25° C. of 5 cSt to 2000 cSt, preferably 10 to 350 cSt, particularly preferably 50 to 100 cSt, such as those obtainable e.g. under the commercial names Dow Corning® 200 or Xiameter PMX from Dow Corning or Xiameter. Further preferred nonvolatile silicone oils are phenyl trimethicones having a kinematic viscosity at 25° C. of 10 to 100 cSt, preferably of 15 to 30 cSt, and cetyl dimethicones.

Natural and synthetic hydrocarbons preferred according to the invention are selected from paraffin oils, isohexadecane, isoeicosane, polyisobutenes, and polydecenes, which are obtainable e.g. under the name Emery® 3004, 3006, 3010 or under the name Nexbase® 2004G from Nestle, as well as 1,3-di-(2-ethylhexyl)cyclohexane.

The compositions according to the invention and used according to the invention optionally include further carrier substances, adjuvants, and active substances.

Aerosol sprays and pump sprays can be present as anhydrous suspensions, water-in-oil emulsion, oil-in-water emulsion, silicone oil-in-water emulsion, water-in-oil microemulsion, oil-in-water microemulsion, silicone oil-in-water microemulsion, alcohol solution, particularly ethanol solution, hydroalcohol solution, particularly solutions having more than 50 wt. % of a water-ethanol mixture, glycol solution, particularly as a solution in 1,2-propylene glycol, glycerol, dipropylene glycol, and (under normal conditions) liquid polyethylene glycols, hydroglycol solution, polyol solution, and as a water-polyol solution. All the aforementioned compositions can be thickened, for example on the basis of fatty acid soaps, dibenzylidene sorbitol, N-acylamino acid amides, 12-hydroxystearic acid, polyacrylates of the carbomer and carbopol type, polyacrylamides, and polysaccharides, which can be chemically and/or physically modified, and particularly by sheet silicates, particularly preferably by montmorillonite, kaolinite, illite, beidellite, nontronite, saponite, hectorite, bentonite, smectite, and talc. The agents can be transparent, translucent, or opaque.

### Lipid or Wax Matrix

The agents according to the invention can preferably contain at least one lipid component or wax component having a melting point >50° C., provided they are not contained in such high amounts that the agents are no longer sprayable. A corresponding total amount is preferably 0.001 to 5 wt. %, particularly preferably 0.01 to 2 wt. %, in each case based on the weight of the total agent without taking into account an optionally present propellant.

Natural vegetable waxes, for example, are preferred according to the invention, for example candelilla wax, carnauba wax, Japan wax, sugar cane wax, ouricury wax, cork wax, sunflower wax, fruit waxes such as orange waxes, lemon waxes, grapefruit wax, and animal waxes, e.g. beeswax, shellac wax, and spermaceti. According to the invention, it can be particularly preferred to use hydrogenated or hardened waxes. Also usable as a wax component are chemically modified waxes, particularly the hard waxes, such as, e.g. montan ester waxes, hydrogenated jojoba waxes, and sasol waxes. Included among the synthetic waxes that are likewise preferred according to the invention are, for example, polyalkylene waxes and polyethylene glycol waxes, C₂₀ to C₄₀ dialkyl esters of dimer acids, C₃₀₋₅₀ alkyl beeswax, and alkyl and alkyl aryl esters of dimer fatty acids.

A particularly preferred wax component is selected from at least one ester of a saturated monovalent C₁₆ to C₆₀ alcohol and a saturated C₈ to C₃₆ monocarboxylic acid. Also included according to the invention are lactides, the cyclic double esters of α-hydroxycarboxylic acids of the corresponding chain length. Esters of fatty acids and long-chain alcohols have proven particularly advantageous for the composition according to the invention since they impart outstanding sensory properties to the antiperspirant composition, and high stability to the stick in total. The esters are made up of saturated branched or unbranched monocarboxylic acids and saturated branched or unbranched monovalent alcohols. Esters of aromatic carboxylic acids or hydroxycarboxylic acids (e.g. 12-hydroxystearic acid) and saturated branched or unbranched alcohols are also usable according to the invention, provided that the wax component has a melting point >50° C. It is particularly preferred to select the wax components from the group of esters of saturated branched or unbranched alkanecarboxylic acids having a chain length of 12 to 24 carbon atoms, and saturated branched or unbranched alcohols having a chain length of 16 to 50 carbon atoms, which have a melting point >50° C. C₁₆₋₃₆ alkyl stearates and C₁₈₋₃₈ alkylhydroxystearoyl stearates, C₂₀₋₄₀ alkyl erucates, and cetearyl behenate can be particularly advantageous as wax component. The wax or the wax components has/have a melting point >50° C., preferably >60° C. A particularly preferred embodiment of the invention contains as a wax component a C₂₀ to C₄₀ alkyl stearate. A further particularly preferred embodiment of the invention contains as a wax component cetearyl behenate, i.e. mixtures of cetyl behenate and stearyl behenate.

Further preferred lipid components or wax components having a melting point >50° C. are triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (Tribehenin), or glyceryl tri-12-hydroxystearate, furthermore synthetic full esters of fatty acids and glycols or polyols having 2 to 6 carbon atoms, provided they have a melting point above 50° C., for example preferably C₁₈ to C₃₆ acid triglycerides (Syncrowax® HGL-C). Hydrogenated castor oil, obtainable e.g. as the commercial product Cutina® HR, is particularly preferred as a wax component according to the invention.

Further preferred lipid components or wax components having a melting point >50° C. are the saturated linear C₁₄ to C₃₆ carboxylic acids, particularly myristic acid, palmitic acid, stearic acid, and behenic acid, as well as mixtures of these compounds.

Particularly preferably, the agents according to the invention additionally contain at least one emulsifying agent and/or at least one surfactant.

Emulsifying agents and surfactants preferably suitable according to the invention are selected from anionic, cationic, nonionic, amphoteric, particularly ampholytic and zwitterionic, emulsifying agents and surfactants.

Surfactants are amphiphilic (bifunctional) compounds that are made up of at least one hydrophobic and at least one hydrophilic molecular part. The hydrophobic group is preferably a hydrocarbon chain having 8 to 28 carbon atoms, which can be saturated or unsaturated, linear or branched. This C₈ to C₂₈ alkyl chain is particularly preferably linear.

The fundamental properties of surfactants and emulsifying agents are oriented absorption on boundary layers, as well as aggregation to micelles and formation of lyotrophic phases.

Anionic surfactants are understood as surfactants having exclusively anionic charges; they contain, for example, carboxyl groups, sulfonic acid groups, or sulfate groups. Particularly preferred anionic surfactants are alkyl sulfates, alkyl ether sulfates, acyl glutamates, and C8 to C24 carboxylic acids, as well as the salts thereof, the so-called soaps.

Cationic surfactants are understood as surfactants having exclusively cationic charges; they contain, for example, quaternary ammonium groups. Cationic surfactants of the quaternary ammonium compound, esterquats, and amidoamine types are preferred. Preferred quaternary ammonium compounds are ammonium halides as well as the imidazolium compounds known by the INCI names Quaternium-27 and Quaternium-83. Further cationic surfactants usable according to the invention are quaternized protein hydrolysates. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Cationic surfactants are contained in the agents preferred according to the invention preferably in proportions of 0.05 to 10 wt. %, based on the total agent without taking into account an optionally present propellant.

Amphoteric surfactants are subdivided into ampholytic surfactants and zwitterionic surfactants Ampholytic surfactants are understood as those surface-active compounds which possess both acidic (for example —COOH or SO₃H groups) and basic hydrophilic groups (for example amino groups), and therefore behave in either acidic or basic fashion depending on the condition. A person skilled in the art understands zwitterionic surfactants as surfactants that carry both a negative and a positive charge in the same molecule.

Examples of preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethylimidazolines, each having 8 to 24 carbon atoms in the alkyl group.

Examples of preferred ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, each having 8 to 24 carbon atoms in the alkyl group.

### Oil-In-Water Emulsifying Agents

The compositions according to the invention that are formulated as an emulsion, particularly as an oil-in-water emulsion, preferably contain at least one nonionic oil-in-water emulsifying agent having an HLB value of more than 7 to 20. These are emulsifying agents commonly known to a person skilled in the art, as listed e.g. in Kirk-Othmer, “Encyclopedia of Chemical Technology,” 3^{rd} ed., 1979, Vol. 8, pp. 913-916. For ethoxylated products, the HLB value is calculated according to the formula HLB=(100−L):5, wherein L is the weight portion of the lipophilic groups, i.e. the fatty alkyl or fatty acyl groups, in the ethylene oxide adducts, expressed as percent by weight.

When selecting nonionic oil-in-water emulsifying agents suitable according to the invention, it is particularly preferred to use a mixture of nonionic oil-in-water emulsifying agents, so that the stability of the agents according to the invention can be optimally adjusted. The individual emulsifying agent components provide in this context a portion of the overall HLB value or average HLB value of the oil-in-water emulsifying agent mixture in accordance with their quantitative proportion in terms of the total amount of oil-in-water emulsifying agents. According to the invention, the average HLB value of the oil-in-water emulsifying agent mixture is to 10 to 19, preferably 12 to 18, and particularly preferably 14 to 17. In order to achieve such average HLB values, it is preferred to combine oil-in-water emulsifying agents from the HLB value ranges 10 to 14, 14 to 16, and optionally 16 to 19 with one another. Of course, the oil-in-water emulsifying agent mixtures can also contain nonionic emulsifying agents having HLB values in the range of >7 to 10 and 19 to 20; such emulsifying agent mixtures can likewise be preferred according to the invention. In another preferred embodiment, however, the antiperspirant compositions according to the invention can contain only a single oil-in-water emulsifying agent having an HLB value in the range of 10 to 19.

Preferred antiperspirant agents according to the invention contain at least one nonionic oil-in-water emulsifying agent that is selected from ethoxylated C₈ to C₂₄ alkanols having an average of 10 to 100 mol ethylene oxide per mol, ethoxylated C₈ to C₂₄ carboxylic acids having an average of 10 to 100 mol ethylene oxide per mol, silicone copolyols having ethylene oxide units or having ethylene oxide and propylene oxide units, alkylmono- and -oligoglycosides having 8 to 22 carbon atoms in the alkyl group and the ethoxylated analogs thereof, ethoxylated sterols, partial esters of polyglycerols having 2 to 10 glycerol units and esterified with 1 to 4 saturated or unsaturated, linear or branched, optionally hydroxylated C₈ to C₃₀ fatty acid groups, provided they have an HLB value of more than 7, as well as mixtures of the aforementioned substances.

The ethoxylated C₈ to C₂₄ alkanols have the formula R¹O(CH₂CH₂O)ₙH, wherein R¹ denotes a linear or branched alkyl group and/or alkenyl group having 8 to 24 carbon atoms, and n denotes the average number of ethylene oxide units per molecule, for numbers from 10 to 100, preferably 10 to 30 mol ethylene oxide per 1 mol capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, as well as the industrial mixtures thereof. Adducts of 10 to 100 mol ethylene oxide to industrial fatty alcohols having 12 to 18 carbon atoms, for example coconut, palm, palm kernel, or tallow fatty alcohol, are also suitable.

The ethoxylated C₈ to C₂₄ carboxylic acids have the formula R¹(OCH₂CH₂)ₙOH, wherein R¹ denotes a linear or branched, saturated or unsaturated acyl group having 8 to 24 carbon atoms, and n denotes the average number of ethylene oxide units per molecule, for numbers from 10 to 100, preferably 10 to 30 mol ethylene oxide per 1 mol caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselic acid, arachidic acid, gadoleic acid, behenic acid, erucic acid, and brassidic acid, as well as the industrial mixtures thereof. Adducts of 10 to 100 mol ethylene oxide to industrial fatty acids having 12 to 18 carbon atoms, for example coconut, palm, palm kernel, or tallow fatty acid, are also suitable. PEG-50 Monostearate, PEG-100 Monostearate, PEG-50 Monooleate, PEG-100 Monooleate, PEG-50 Monolaurate, and PEG-100 Monolaurate are particularly preferred.

It is particularly preferred to use C₁₂ to C₁₈ alkanols or C₁₂ to C₁₈ carboxylic acids each having 10 to 30 units ethylene oxide per molecule, as well as mixtures of these substances, particularly Ceteth-12, Ceteth-20, Ceteth-30, Steareth-12, Steareth-20, Steareth-30, Laureth-12, and Beheneth-20.

C₈ to C₂₂ alkylmono- and -oligoglycosides are also preferably used. C₈ to C₂₂ alkylmono- and -oligoglycosides are known, commercially available surfactants and emulsifying agents. They are manufactured particularly by reacting glucose or oligosaccharides with primary alcohols having 8 to 22 carbon atoms. With regard to the glycoside group, both monoglycosides, in which a cyclic sugar group is bound glycosidically to the fatty alcohol, and oligomeric glycosides having a degree of oligomerization up to approximately 8, preferably 1 to 2, are suitable. The degree of oligomerization is a statistical average that is based on a homolog distribution that is usual for industrial products of this kind Products that are obtainable under the name Plantacare® contain a glucosidically bound C₈ to C₁₆ alkyl group on an oligoglucoside group, the average degree of oligomerization of which is about 1 to 2, particularly about 1.1 to 1.4. Particularly preferred C₈ to C₂₂ alkyl mono- and -oligoglycosides are selected from octyl glucoside, decyl glucoside, lauryl glucoside, palmityl glucoside, isostearyl glucoside, stearyl glucoside, arachidyl glucoside, and behenyl glucoside, as well as mixtures thereof. The acyl glucamides derived from glucamine are also suitable as nonionic oil-in-water emulsifying agents.

Ethoxylated sterols, particularly ethoxylated soy sterols, are also oil-in-water emulsifying agents suitable according to the invention. The degree of ethoxylation can be greater than 5, preferably at least 10, in order to exhibit an HLB value greater than 7 to 20. Suitable commercial products are, for example, PEG-10 Soy Sterol, PEG-16 Soy Sterol, and PEG-25 Soy Sterol.

It is further preferred to use partial esters of polyglycerols having 2 to 10 glycerol units and esterified with 1 to 4 saturated or unsaturated, linear or branched, optionally hydroxylated C₈ to C₃₀ fatty acid groups, provided they have an HLB value in the range of more than 7 to 20. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate, and decaglycerol trihydroxystearate are particularly preferred.

Particularly preferred antiperspirant agents according to the invention are characterized in that the nonionic oil-in-water emulsifying agent is included in a total amount of 0.01 to 10 wt. %, particularly preferably 0.1 to 4 wt. %, and extraordinarily preferably 0.5 to 3 wt. %, based on the total weight of the propellant-free agent.

### Water-In-Oil Emulsifying Agents

Compositions preferred according to the invention that are formulated as an emulsion preferably furthermore contain at least one nonionic water-in-oil emulsifying agent having an HLB value greater than 1.0 and less than or equal to 7.0, selected from mono- and diesters of ethylene glycol and the mono-, di-, tri-, and tetraesters of pentaerythritol with linear saturated fatty acids having 12 to 30, particularly 14 to 22 carbon atoms, which can be hydroxylated, as well as mixtures thereof, as consistency agents and/or water binders. The mono- and diesters are preferred according to the invention. C₁₂ to C₃₀ fatty acid groups preferred according to the invention are selected from lauric acid, myristic acid, palmitic acid, stearic acid, arachidic acid, and behenic acid groups; the stearic acid group is particularly preferred. Nonionic water-in-oil emulsifying agents particularly preferred according to the invention having an HLB value greater than 1.0 and less than or equal to 7.0 are selected from pentaerythrityl monostearate, pentaerythrityl distearate, pentaerythrityl tristearate, pentaerythrityl tetrastearate, ethylene glycol monostearate, ethylene glycol distearate, and mixtures thereof. Water-in-oil emulsifying agents particularly preferred according to the invention having an HLB value greater than 1.0 and less than or equal to 7.0 are obtainable, for example, as the commercial products Cutina® PES (INCI: Pentaerythrityl Distearate), Cutina® AGS (INCI: Glycol Distearate), or Cutina® EGMS (INCI: Glycol Stearate). These commercial products are already mixtures of mono- and diesters (tri- and tetraesters are also contained in the pentaerythrityl esters). It can be preferred according to the invention to use only a single water-in-oil emulsifying agent. In another preferred embodiment, the compositions according to the invention contain mixtures, particularly industrial mixtures, of at least two water-in-oil emulsifying agents. An industrial mixture is understood, for example, as a commercial product such as Cutina® PES.

Besides the aforementioned water-in-oil emulsifying agents based on ethylene glycol esters or pentaerythrityl esters, at least one further nonionic water-in-oil emulsifying agent having an HLB value greater than 1.0 and less than or equal to 7.0 can also be contained in a preferred embodiment, the portion of which in terms of the total weight of nonionic water-in-oil emulsifying agents having an HLB value greater than 1.0 and less than or equal to 7.0 should, however, not be greater than 80%. In a particularly preferred embodiment, the compositions according to the invention contain the at least one additional water-in-oil emulsifying agent having an HLB value greater than 1.0 and less than or equal to 7.0 at a weight proportion of only a maximum of 10% or are free of additional water-in-oil emulsifying agents. Some of these additional suitable emulsifying agents are listed, for example, in Kirk-Othmer, “Encyclopedia of Chemical Technology,” 3^{rd} ed., 1979, Vol. 8, p. 913. As already mentioned, the HLB value can also be calculated for ethoxylated adducts.

The following are preferably suitable as water-in-oil emulsifying agents:

linear saturated alkanols having 12 to 30 carbon atoms, particularly having 16 to 22 carbon atoms, particularly cetyl alcohol, stearyl alcohol, arachidyl alcohol, behenyl alcohol, and lanolin alcohol, or mixtures of these alcohols such as those obtainable from the industrial hydrogenation of vegetable and animal fatty acids;

esters and, particularly, partial esters of a polyol having 3 to 6 carbon atoms and linear saturated and unsaturated fatty acids having 12 to 30, particularly 14 to 22 carbon atoms, which can be hydroxylated. Such esters or partial esters are, e.g., mono- and diesters of glycerol or the monoesters of propylene glycol with linear saturated and unsaturated C₁₂ to C₃₀ carboxylic acids, which can be hydroxylated, particularly those with palmitic and stearic acid, sorbitan mono-, di-, or triesters of linear saturated and unsaturated C₁₂ to C₃₀ carboxylic acids, which can be hydroxylated, particularly those of myristic acid, palmitic acid, stearic acid, or of mixtures of these fatty acids, and methylglucose mono- and diesters of linear saturated and unsaturated C₁₂ to C₃₀ carboxylic acids, which can be hydroxylated;

sterols, i.e. steroids that carry a hydroxyl group on the C3 atom of the steroid skeleton and are isolated both from animal tissue (zoosterols, e.g. cholesterol, lanosterol) and from plants (phytosterols, e.g. ergosterol, stigmasterol, sitosterol) and from fungi and yeasts (mycosterols), and can have a low degree of ethoxylation (1 to 5 EO);

alkanols and carboxylic acids each having 8 to 24 carbon atoms, particularly having 16 to 22 carbon atoms, in the alkyl group and 1 to 4 ethylene oxide units per molecule, which have an HLB value greater than 1.0 and less than or equal to 7.0;

glycerol monoethers of saturated and/or unsaturated, branched and/or unbranched alcohols having a chain length of 8 to 30, particularly 12 to 18 carbon atoms;

partial esters of polyglycerols having n=2 to 10 glycerol units and esterified with 1 to 5 saturated or unsaturated, linear or branched, optionally hydroxylated C₈ to C₃₀ fatty acid groups, provided they have an HLB value less than or equal to 7;

as well as mixtures of the aforementioned substances.

It can be preferred according to the invention to use only a single additional water-in-oil emulsifying agent. In another preferred embodiment, the compositions according to the invention contain mixtures, particularly industrial mixtures, of at least two additional water-in-oil emulsifying agents. An industrial mixture is understood, for example, as a commercial product such as Cutina® GMS, which is a mixture of glyceryl monostearate and glyceryl distearate.

Additional water-in-oil emulsifying agents usable particularly advantageously are stearyl alcohol, cetyl alcohol, glyceryl monostearate, particularly in the form of the commercial products Cutina® GMS and Cutina® MD (ex BASF), glyceryl distearate, glyceryl monocaprinate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol(2) stearyl ether (Steareth-2), Steareth-5, Oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, Soy Sterol, PEG-1 Soy Sterol, PEG-5 Soy Sterol, PEG-2 monolaurate, and PEG-2 monostearate.

Particularly preferred antiperspirant compositions according to the invention are characterized in that at least one water-in-oil emulsifying agent is contained in a total amount of 0.01 to 10 wt. %, particularly preferably 0.1 to 4 wt. %, and extraordinarily preferably 0.5 to 3 wt. %, based on the total weight of the propellant-free agent.

The HLB values can be calculated according to Griffin, as presented or tabulated, for example, in the “RÖMPP Chemie Lexikon”, particularly in the online version of November 2003, and in the handbooks of Fiedler, Kirk-Othmer, and Janistyn (H. Janistyn, Handbuch der Kosmetika and Riechstoffe, Huthig-Verlag Heidelberg, 3^{rd} ed. 1978, Vol. 1, p. 470, and Vol. 3, pp. 68-78) cited therein under the keyword “HLB system.” If there are different specifications in the literature regarding the HLB value of a substance, that HLB value which is closest to the value calculated according to Griffin should be used for the teaching according to the invention. If an unequivocal HLB value cannot be ascertained in this manner, the HLB value indicated by the manufacturer of the emulsifying agent is to be used for the teaching according to the invention. If this is also not possible, the HLB value is to be ascertained experimentally.

Further preferred compositions according to the invention are characterized in that the total content of nonionic and ionic emulsifying agents and/or surfactants having an HLB value above 8 is at most 20 wt. %, preferably at most 15 wt. %, particularly preferably at most 10 wt. %, more particularly preferably at most 7 wt. %, further particularly preferably at most 4 wt. %, and extraordinarily preferably at most 3 wt. %, in each case based on the total agent according to the invention without taking into account optionally present propellants.

Compositions particularly preferred according to the invention that are prepared as a water-in-oil emulsion preferably furthermore contain at least one water-in-oil emulsifying agent. The at least one water-in-oil emulsifying agent is contained preferably in an amount of 0.1 to 8 wt. %, particularly preferably 1.0 to 2.5 wt. %, in each case based on the total weight of the agent without taking into account optionally present propellants.

A group of water-in-oil emulsifying agents particularly preferred according to the invention are the poly-(C₂-C₃)alkylene glycol-modified silicones, the previous INCI name of which was Dimethicone Copolyol, having the current INCI names PEG-x Dimethicone (with x=2 to 20, preferably 3 to 17, particularly preferably 11 to 12), Bis-PEG-y Dimethicone (with y=3 to 25, preferably 4 to 20), PEG/PPG a/b Dimethicone (with a and b mutually independently denoting numbers from 2 to 30, preferably 3 to 30, and particularly preferably 12 to 20, particularly 14 to 18), Bis-PEG/PPG-c/d Dimethicone (with c and d mutually independently denoting numbers from 10 to 25, preferably 14 to 20, and particularly preferably 14 to 16), and Bis-PEG/PPG-e/f PEG/PPG g/h Dimethicone (with e, f, g, and h mutually independently denoting numbers from 10 to 20, preferably 14 to 18, and particularly preferably 16). PEG/PPG-18/18 Dimethicone, which is commercially available in a 1:9 mixture with Cyclomethicone as DC 3225 C or DC 5225 C; as a 25:75 mixture with Dimethicone as DC 5227, PEG/PPG-4/12 Dimethicone, which is obtainable under the designation Abil B 8852, and Bis-PEG/PPG-14/14 Dimethicone, which is commercially available in a mixture with Cyclomethicone as Abil EM 97 (Goldschmidt), Bis-PEG/PPG-20/20 Dimethicone, which is obtainable under the designation Abil B 8832, PEG/PPG-5/3 Trisiloxane (Silsoft 305), and PEG/PPG-20/23 Dimethicone (Silsoft 430 and Silsoft 440) are particularly preferred.

Further W/O emulsifying agents preferred according to the invention are poly-(C₂-C₃)alkylene glycol-modified silicones that are hydrophobically modified with C₄ to C₁₈ alkyl groups, particularly preferably Cetyl PEG/PPG-10/1 Dimethicone (formerly: Cetyl Dimethicone Copolyol, obtainable as Abil EM 90 or in a mixture of polyglyceryl-4-isostearate, Cetyl PEG/PPG-10/1 Dimethicone, and hexyl laurate under the trade name Abil WE 09), also Alkyl Methicone Copolyols.

Compositions particularly preferred according to the invention furthermore contain preferably at least one skin-cooling active substance. Skin-cooling active substances suitable according to the invention are, for example, menthol, isopulegol, and menthol derivatives, e.g. menthyl lactate, menthyl glycolate, menthyl ethyl oxamate, menthylpyrrolidonecarboxylic acid, menthyl methyl ether, menthoxypropanediol, menthone glycerol acetal (9-methyl-6-(1-methylethyl)-1,4-dioxaspiro (4.5)decane-2-methanol), monomenthyl succinate, and 2-hydroxymethyl-3,5,5-trimethylcyclohexanol. Menthol, isopulegol, menthyl lactate, menthoxypropanediol, and menthylpyrrolidonecarboxylic acid as well as mixtures of these substances, particularly mixtures of menthol and menthyl lactate, menthol, menthol glycolate and menthyl lactate, menthol and menthoxypropanediol, or menthol and isopulegol are preferred as skin-cooling active substances.

It is particularly preferred according to the invention that at least one skin-cooling active substance be contained in a total amount of 0.01 to 2 wt. %, particularly preferably 0.02 to 0.5 wt. %, and extraordinarily preferably 0.05 to 0.2 wt. %, in each case based on the total weight of the agent without taking into account optionally present propellants.

Compositions particularly preferred according to the invention, which are prepared as propellant-driven aerosol, contain at least one propellant. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes, each individually as well as in combination. Hydrophilic propellant gases, such as carbon dioxide, can also be used advantageously in accordance with the present invention if the amount of hydrophilic gases is selected to be low and a lipophilic propellant gas (e.g. propane, butane) is abundantly present. Propane, n-butane, isobutane as well as mixtures of these propellant gases are particularly preferred. The use of n-butane as the only propellant gas has shown that it can be particularly preferable according to the invention.

The amount of propellants is preferably 20 to 95 wt. %, particularly preferably 30 to 85 wt. %, and extraordinarily preferably 40 to 75 wt. %, in each case based on the total weight of the preparation, consisting of the composition according to the invention (=agent according to the invention) and the propellant.

Vessels made of metal (aluminum, tinplate, tin), protected or shatterproof plastic, or glass coated with plastic on the outside, are possible pressurized gas containers, for the selection of which compressive and fracture strength, corrosion resistance, ability to be filled easily as well as esthetic aspects, handiness, printability, etc. are taken into consideration. Specific protective inner coatings ensure corrosion resistance against the composition according to the invention.

Preferred compositions according to the invention furthermore contain at least one water-soluble polyvalent C₂ to C₉ alkanol having 2 to 6 hydroxyl groups and/or at least one water-soluble polyethylene glycol having 3 to 20 ethylene oxide units, as well as mixtures thereof. These components are preferably selected from 1,2-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of cis- and trans-1,4-dimethylolcyclohexane, as well as mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18, and PEG-20 as well as mixtures thereof, wherein PEG-3 to PEG-8 are preferred.

Preferred antiperspirant agents according to the invention contain at least one water-soluble polyvalent C₂ to C₉ alkanol having 2 to 6 hydroxyl groups and/or at least one water-soluble polyethylene glycol having 3 to 20 ethylene oxide units, which is selected from 1,2-propylene glycol, 2-methyl-1,3-propanediol, glycerol, 1,2-butylene glycol, 1,3-butylene glycol, 1,4-butylene glycol, pentylene glycols such as 1,2-pentane diol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of cis- and trans-1,4-dimethylolcyclohexane, as well as mixtures of the aforementioned substances.

Particularly preferred antiperspirant agents according to the invention contain at least one water-soluble polyvalent C₂ to C₉ alkanol having 2 to 6 hydroxyl groups and/or at least one water-soluble polyethylene glycol having 3 to 20 ethylene oxide units, in a total amount of 0.1 to 30 wt. %, preferably 1 to 20 wt. %, particularly preferably 5 to 15 wt. %, in each case based on the total weight of the agent without taking into account an optionally present propellant.

Particularly preferred antiperspirant agents according to the invention are characterized in that at least one lipid component or wax component having a melting point in the range of 25 to <50° C., selected from coconut fatty acid glycerol mono-, di-, and triesters, Butyrospermum parkii (shea butter), and esters of saturated monovalent C₈ to C₁₈ alcohols with saturated C₁₂ to C₁₈ monocarboxylic acids, as well as mixtures of these substances, is contained. Commercial products having the INCI name Cocoglycerides are particularly preferred, particularly the commercial products Novata® (ex BASF), particularly preferably Novata® AB, a mixture of C₁₂ to C₁₈ mono-, di-, and triglycerides that melts in the range of 30 to 32° C., as well as the products of the Softisan series (Sasol Germany GmbH) having the INCI name Hydrogenated Cocoglycerides, particularly Softisan 100, 133, 134, 138, 142. Further preferred esters of saturated monovalent C₁₂ to C₁₈ alcohols with saturated C₁₂ to C₁₈ monocarboxylic acids are stearyl laurate, cetearyl stearate (e.g. Crodamol® CSS), cetyl palmitate (e.g. Cutina® CP), and myristyl myristate (e.g. Cetiol® MM).

Particularly preferred antiperspirant agents according to the invention further contain at least one odorous substance. The definition of an odorous substance for purposes of the present application corresponds to the definition common to a person skilled in the art, as can be found in the RÖMPP Chemie Lexikon as of December 2007. According thereto, an odorous substance is a chemical compound having an odor and/or taste that excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule. In order to distinguish volatile low-molecular-weight substances that are commonly, and also in accordance with the present application, regarded and utilized not as odorous substance but instead principally as solvent, for example ethanol, propanol, isopropanol, and acetone, from odorous substances according to the invention, odorous substances according to the invention have a molar mass of 74 to 300 g/mol, contain at least one osmophoric group in the molecule, and have an odor and/or taste, i.e. they excite the receptors of the hair cells of the olfactory system.

Perfumes, perfume oils, or perfume oil constituents can be used as odorous substances. According to the invention, perfume oils or scents can be individual odorous substance compounds, e.g. synthetic products of the ester, ether, aldehyde, ketone, alcohol, and hydrocarbon types. Odorous substance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmecyclate. The ethers include, for example, benzyl ethyl ether and ambroxan; the aldehydes include, for example, the linear alkanals having 8 to 18 carbon atoms, citral, citronellal, citronellyloxyacetaldehyde, cyclamenaldehyde, lilial, and bourgeonal; the ketones include, for example, the ionones, alpha-isomethylionone and methyl cedryl ketone; the alcohols include anethol, citronellol, eugenol, geraniol, linalool, phenylethyl alcohol and terpineol; and the hydrocarbons include principally the terpenes such as limonene and pinene. Preferably, however, mixtures of different odorous substances that together produce an attractive scent are used.

Particularly preferred antiperspirant agents according to the invention contain at least one odorous substance component in a total amount of 0.00001 to 10 wt. %, preferably 0.5 to 7 wt. %, extraordinarily preferably 1 to 6 wt. %, in each case based on the total weight of the agent without taking into account an optionally present propellant.

In a preferred embodiment according to the invention, the at least one perspiration-inhibiting aluminum salt is suspended undissolved in at least one oil that is liquid under normal conditions. For better applicability, at least one lipophilic thickening agent as suspension aid is further added to this suspension. Further preferred compositions according to the invention are thus characterized in that they contain at least one lipophilic thickening agent. Lipophilic thickening agents preferred according to the invention are selected from hydrophobized clay minerals and pyrogenic silicic acids. Among those, hydrophobized clay minerals are particularly preferred.

Preferred hydrophobized clay minerals are selected from hydrophobized montmorillonites, hydrophobized hectorites, and hydrophobized bentonites, particularly preferably from disteardimonium hectorite, stearalconium hectorite, Quaternium-18 hectorite, and Quaternium-18 bentonite. The commercially available thickening agents provide these hydrophobized clay minerals in the form of a gel in an oil component, preferably in cyclomethicone and/or a non-silicone oil component, e.g. propylene carbonate. The gelling is generated by the addition of small amounts of activators, particularly such as ethanol or propylene carbonate, but also water. Such gels are available, for example, under the trade name Bentone® or Thixogel. Preferred compositions according to the invention contain at least one activator in a total amount of 0.1 to 3 wt. %, preferably 0.3 to 1.6 wt. %, in each case based on the total weight of the propellant-free composition according to the invention. Further preferred compositions according to the invention contain at least one activator, selected from ethanol, propylene carbonate, and water, as well as mixtures therefrom, in a total amount of 0.1 to 3 wt. %, preferably 0.3 to 1.6 wt. %, in each case based on the total weight of the propellant-free composition according to the invention. Compositions preferred according to the invention are characterized in that they contain at least one hydrophobized clay mineral in a total amount of 0.5 to 10 wt. %, preferably 1 to 7 wt. %, particularly preferably 2 to 6 wt. %, extraordinarily preferably 3 to 5 wt. %, in each case based on the total weight of the propellant-free composition according to the invention.

Further lipophilic thickening agents preferred according to the invention are selected from pyrogenic silicic acids, e.g. the commercial products of the Aerosil® series by Evonik Degussa. Hydrophobized pyrogenic silicic acids are particularly preferred, particularly preferably silica silylate and silica dimethyl silylate.

Compositions preferred according to the invention are characterized in that they contain at least one pyrogenic silicic acid, preferably at least one hydrophobized pyrogenic silicic acid in a total amount of 0.5 to 10 wt. %, preferably 0.8 to 5 wt. %, particularly preferably 1 to 4 wt. %, extraordinarily preferably 1.5 to 2 wt. %, in each case based on the total weight of the propellant-free composition according to the invention.

Further compositions preferred according to the invention are characterized in that they contain at least one hydrophobized pyrogenic silicic acid and at least one hydrophilic silicic acid.

A further subject matter of the present application is a method for nontherapeutic, cosmetic perspiration-inhibiting treatment of the body, in which a perspiration-inhibiting cosmetic agent, which contains, in a cosmetically compatible carrier, at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the composition, and in addition thereto methanesulphonic acid and/or at least one physiologically compatible salt of methanesulphonic acid, preferably in a total amount of 0.05 to 5 wt. %, more preferably 0.1 to 3 wt. %, particularly preferably 0.5 to 2.5 wt. %, extraordinarily preferably 1 to 2 wt. %, is applied to the skin, particularly to the skin of the armpits, wherein the specifications in wt. % refer in each case to the total weight of the agent without taking into account an optionally present propellant.

The statements made with regard to the agents according to the invention apply, mutatis mutandis, to preferred embodiments of the method according to the invention.

A further subject matter of the present application is a method for preventing and/or reducing textile discolorations and/or textile stains, wherein the method comprises the following method steps:
(a) producing a perspiration-inhibiting cosmetic agent by mixing at least one perspiration-inhibiting zirconium-free aluminum salt in a total amount of 2 to 40 wt. %, preferably 8 to 35 wt. %, particularly preferably 10 to 28 wt. %, and extraordinarily preferably 12 to 20 wt. %, wherein the specifications in wt. % refer to the total weight of the active substance (USP), free of water of crystallization and free of ligands, in the agent, with a cosmetically compatible carrier and with methanesulphonic acid of the following formula (MS-1)

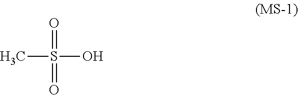

or at least one physiologically compatible salt of methanesulphonic acid, wherein methanesulphonic acid or the salt(s) thereof is/are contained in a total amount of 0.05 to 5 wt. %, preferably 0.1 to 3 wt. %, particularly preferably 0.5 to 2.5 wt. %, extraordinarily preferably 1 to 2 wt. %, wherein the specifications in wt. % refer in each case to the total weight of the propellant-free agent;
(b) filling the agent in a packaging selected from a pump spray container, a squeeze container, and a spray can containing at least one propellant;
(c) applying the perspiration-inhibiting cosmetic agent to the skin, particularly to the skin of the armpits;
(d) wearing a textile garment over the treated skin; and
(e) washing the textile garment, particularly repeatedly washing the textile garment,
   wherein no, or reduced, textile discolorations and/or textile stains occur after washing, particularly after repeated washing.

The statements made with regard to the agents and products according to the invention apply, mutatis mutandis, with regard to preferred embodiments of the method according to the invention for preventing and/or reducing textile discolorations and/or textile stains.

### Experimental Section

Anhydrous Antiperspirant Compositions

An oily suspension consisting of 14.3 wt. % activated aluminum chlorohydrate, 2 wt. % of the methanesulphonic acid of formula (MS-1), used according to the present invention, to be tested, as well as 65.9 wt. % 2-ethylhexyl palmitate, 5.4 wt. % triethyl citrate, 3.9 wt. % Bentone 38 V CG, 7.2 wt. % perfume oil, and 1.3 wt. % propylene carbonate, was produced as a test product according to the present invention (E-1). Such a suspension is, inter alia, representative of anhydrous antiperspirant roll-ons and anhydrous antiperspirant wax sticks.

A suspension consisting of 14.3 wt. % activated aluminum chlorohydrate and 67.9 wt. % 2-ethylhexyl palmitate, 5.4 wt. % triethyl citrate, 3.9 wt. % Bentone 38 V CG, 1.3 wt. % propylene carbonate and 7.2 wt. % perfume oil, was produced as a comparison product not according to the invention (V-1).

TABLE 1
Test and comparison products used (quantities indicated in wt. %)

| | V-1 | E-1 |
|---|---|---|
| Activated aluminum chlorohydrate (AACH) | 14.3 | 14.3 |
| 2-Ethylhexyl palmitate | 67.9 | 65.9 |
| Triethyl citrate | 5.4 | 5.4 |
| Bentone 38 V CG | 3.9 | 3.9 |
| Propylene carbonate | 1.3 | 1.3 |
| Perfume | 7.2 | 7.2 |
| Methanesulphonic acid | — | 2 |

### Test Execution

0.3 gram of the respective test product or comparison product was applied directly onto a 10×10 cm² piece of light-blue cotton material (polo jersey, woven) that was stitched onto a waffle pique towel. After one hour of waiting time, 1 ml of an artificial perspiration mixture (MgCl₂, CaCl₂, KCl, NaCl, Na₂SO₄, NaH₂PO₄, glycine, glucose, lactic acid, urea; pH 5.2) was applied, and after a 24 hour waiting time (aging), the textile was washed in a standardized common household washing process (Miele W 1714) and mechanically dried (Miele T 7644C).

TABLE 2
Further conditions of the washing tests

| | |
|---|---|
| Washing machine load: | 3.5 kg |
| Water volume: | 17 liters |
| Temperature: | 40° C. |
| Main washing cycle time: | 1 h |
| Prewash: | None |
| Rinse: | 4x |
| Detergent: | Spee color gel |
| | Batch: HH06.1.1UWM1.08:58 |
| Weighed portion detergent: | 75 ml (70 g) |
| Fabric softener: | None |
| Dryer program: | Extra dry - cotton |

Product application and washing were repeated a total of eight times using the same textile (eight soiling/washing cycles). Textile soiling was evaluated visually by trained laboratory assistants on the basis of reference examples. The scale ranged from 0 (no stains) to 4 (very heavy staining). The evaluation was performed directly after completion of the washing series.

TABLE 3
Results of visual residue evaluation

| | after 8th wash | | |
|---|---|---|---|
| Product | White | greasy | yellow |
| V-1 | 0 | 4 | 0 |
| E-1 | 0 | 2.5 | 0 |

The test product E-1 according to the invention, having 2 wt. % methanesulphonic acid, exhibited a significantly reduced formation of greasy stains on light-blue textile (Table 3) as compared with the comparison formulation V-1 having no methanesulphonic acid (Table 1).

A further washing test with an anhydrous antiperspirant aerosol active substance suspension according to Table 9, example 4.1 (see below) showed a significant reduction of white stains on light-blue cotton material.

### Hydrous Antiperspirant Compositions

An aqueous solution consisting of 20 wt. % aluminum chlorohydrate, 2 wt. % methanesulphonic acid, as well as 78 wt. % water, was produced as a test product according to the invention (E-2). Such a solution is representative, inter alia, of hydrous antiperspirant emulsions (antiperspirant emulsion sprays).

A solution consisting of 20 wt. % aluminum chlorohydrate and 80 wt. % water was used as a comparison product not according to the invention (V).

TABLE 4
Test and comparison products used (quantities indicated in wt. %)

| | V-2 | E-2 |
|---|---|---|
| Aluminum chlorohydrate (ACH) | 20 | 20 |
| Water | 80 | 78 |
| Methanesulphonic acid | — | 2 |

The washing tests were carried out analogously to the test series using the anhydrous compositions.

TABLE 5
Results of visual residue evaluation

| | after 8th wash | | |
|---|---|---|---|
| Product | white | greasy | yellow |
| V-2 | 4 | 0 | 0 |
| E-2 | 2 | 0 | 0 |

The test product according to the invention E-2, having 2 wt. % methanesulphonic acid, exhibited a significantly reduced formation of white stains on light-blue textile (Table 5) as compared with the comparison formulation V-2 having no methanesulphonic acid (Table 4).

Hydrous Antiperspirant Oil-In-Water Emulsion

An oil-in-water emulsion of the following composition E-3 was produced as a test product according to the invention:

TABLE 6
Test emulsion (O/W) according and not according
to the invention; quantities in wt. %

| | V-3 | E-3 |
|---|---|---|
| ALUMINUM CHLOROHYDRATE | 20.0 | 20.0 |
| STEARETH-2 | 2.4 | 2.4 |
| STEARETH-21 | 1.5 | 1.5 |
| PERFUME | 1.0 | 1.0 |
| PPG-15 STEARYL ETHER | 0.5 | 0.5 |
| ALUMINUM STARCH OCTENYLSUCCINATE | 0.1 | 0.1 |
| Isopropyl myristate | 0.1 | 0.1 |
| Vitamin E acetate | 0.05 | 0.05 |
| EDTA powder | 0.095 | 0.095 |
| Methanesulphonic acid | — | 0.5 |
| Water | to 100.0 | to 100.0 |

A solution of this kind is representative of hydrous antiperspirant emulsions (antiperspirant emulsion sprays).

The washing tests were carried out similarly to the test series presented above; however, more washing cycles were carried out and the textiles were aged by being stored for 14 days. This allows for the development of yellow stains to be simulated and evaluated.

### Test Execution

0.3 gram of the respective test product or comparison product was applied directly onto a 9.5×9.5 cm² piece of white cotton material (T-shirt material, knitted) that was stitched onto a waffle pique towel. After one hour of waiting time, 1 ml of an artificial perspiration mixture (MgCl₂, CaCl₂, KCl, NaCl, Na₂SO₄, NaH₂PO₄, glycine, glucose, lactic acid, urea; pH 5.2) was applied, and after a 24 hour waiting time (aging), the textile was washed (Miele W 1714) and mechanically dried (Miele T 7644C).

TABLE 7
Further conditions of the washing tests

| | |
|---|---|
| Washing machine load: | 3.5 kg |
| Water volume: | 17 liters |
| Temperature: | 40° C. |
| Main washing cycle time: | 1 h |
| Prewash: | None |
| Rinse: | 4x |
| Detergent: | Persil Gold universal powder |
| | with brightness formula |
| | Wero 2020110023 |
| Weighed portion detergent: | 135 ml (80 g) |
| Fabric softener: | None |
| Dryer program: | Extra dry - cotton |

Product application and washing were repeated for a total of fourteen times using the same textile. Textile soiling was evaluated visually by trained laboratory assistants on the basis of reference examples. The scale ranged from 0 (no stains) to 4 (very heavy staining). The evaluation was performed 14 days after completion of the washing series.

TABLE 8
Results of visual residue evaluation

| | after 14 washes + 14 days aging | | |
|---|---|---|---|
| Product | white | greasy | yellow |
| V-3 | 0 | 0 | 2 |
| E-3 | 0 | 0 | 1 |

The test product according to the invention E-3, having 0.5 wt. % methanesulphonic acid, exhibited a significantly reduced formation of yellow stains on white textile (Table 8) as compared with the comparison formulation V-3 having no methanesulphonic acid (Table 6).

### Embodiments

The following formulation examples are intended to explain the subject matter of the invention without limiting it thereto.

TABLE 9
Translucent antiperspirant microemulsions sprayable
as pump sprays (quantities indicated in wt. %)

| | 1.1 | 1.2 | 1.3 | 1.4 | 1.5 | 1.6 |
|---|---|---|---|---|---|---|
| Plantaren ® 1200 | 1.71 | 1.71 | — | 1.71 | 1.71 | — |
| Plantaren ® 2000 | 1.14 | 1.39 | 2.40 | 1.14 | 1.39 | 2.40 |
| Glycerol monooleate | 0.71 | 0.71 | — | 0.71 | 0.71 | — |
| Dioctyl ether | 4.00 | 4.00 | 0.09 | 4.00 | 4.00 | 0.09 |
| Octyl dodecanol | 1.00 | 1.00 | 0.02 | 1.00 | 1.00 | 0.02 |
| Perfume oil | 1.00 | 1.00 | 1.00 | 1.00 | 1.00 | 1.00 |
| Aluminum | 8.00 | 5.00 | 5.00 | 15.00 | 10.00 | 12.00 |
| chlorohydrate | | | | | | |
| 1,2-Propylene glycol | 5.00 | 5.00 | — | 5.00 | 5.00 | 5.00 |
| Glycerol | — | — | 5.00 | — | — | — |
| 2-Benzylheptan-1-ol | 0.5 | — | — | 0.5 | 0.5 | — |
| Triethyl citrate | — | 0.5 | 0.5 | 0.2 | — | — |
| Triclosan | — | — | — | — | — | 0.5 |
| Methanesulphonic | 1.0 | 0.5 | 2.0 | 2.5 | 0.5 | 3.0 |
| acid | | | | | | |
| Water | to 100 | to 100 | to 100 | to 100 | to 100 | to 100 |

TABLE 10
Water-in-oil emulsions according to the invention (all
quantities indicated in wt. %, based on the total weight
of the water-in-oil emulsion without propellant)

| | 2.1 | 2.2 | 2.3 |
|---|---|---|---|
| Aluminum chlorohydrate | 33 | 33 | 33 |
| C₁₀ to C₁₃ isoalkane | 8.9 | 8.9 | 8.9 |
| PEG/PPG-18/18 Dimethicone* | 1.4 | 1.4 | 1.4 |
| Isoceteth-20 | 0.50 | 0.50 | 0.50 |
| Dimethicone* | 4.2 | 4.2 | 4.2 |
| Isopropyl myristate | 9.0 | 9.0 | 9.0 |
| 1,2 propanediol | 7.0 | 25 | 25 |
| Phenoxyethanol | 0.50 | 0.50 | 0.50 |
| Perfume | 2.5 | 2.5 | 2.5 |
| Methanesulphonic acid | 2.0 | 0.5 | 3.0 |
| L-menthol | 0.4 | 0.3 | — |
| Trans-anethol | — | 0.3 | — |
| Eucalyptol | — | 0.3 | — |
| Water | to 100 | to 100 | to 100 |

*ex Dow Corning ES-5227 DM

The perspiration-inhibiting water-in-oil emulsions according to the invention 2.1, 2.2, and 2.3 are filled into an aluminum spray can coated on the inside with epoxy phenolic lacquer, at a weight ratio of propellant (butane/propane/isobutane mixture) to emulsion of 80:20 or 85:15 or 60:40 or 90:10.

The example compositions according to the invention are sprayed onto the skin of the armpit.

TABLE 11
Antiperspirant sprays in the form of a water-in-
oil emulsion (quantities indicated in wt. %, based
on the propellant-containing composition)

| | 3.1 | 3.3 |
|---|---|---|
| Aluminum chlorohydrate 50% in water (Locron L) | 10.0 | 10.0 |
| Pionier 2094 | 1.7 | 1.7 |
| Dow Corning ES-5227 DM Formulation Aid | 1.0 | 1.0 |
| 1,2-Propylene glycol | 5.0 | 5.0 |
| Methanesulphonic acid | 2.0 | 0.5 |
| EDTA | — | 0.01 |
| Propane | 12.0 | 12.0 |
| Butane | 68.0 | 68.0 |
| Perfume | 1.0 | 1.0 |
| Isopropyl myristate | to 100 | to 100 |

TABLE 12
Suspensions for spraying as antiperspirant spray

| | 4.1 | 4.2 |
|---|---|---|
| Perfume | 7.00 | 7.00 |
| Aluminum chlorohydrate (activated) | 30.00 | 35.00 |
| Methanesulphonic acid | 2.00 | 0.5 |
| Isopropyl palmitate | 5.00 | 5.00 |
| Disteardimonium hectorite | 4.50 | 2.5 |
| Propylene carbonate | 1.50 | 0.9 |
| Cyclopentasiloxane | to 100 | to 100 |

The compositions according to the invention 4.1 and 4.2 were filled into spray cans made of aluminum lacquered on the inside, and an isobutane/butane/propane propellant mixture at a weight ratio suspension to propellant of 25:75, 22:78, 20:80, and 15:85 was applied thereto.

List of Raw Materials Used

| | | Supplier/ |
|---|---|---|
| Component | INCI | Manufacturer |
| Dow Corning ES-5227 | Dimethicone, PEG/PPG- | Dow Corning |
| DM Formulation Aid | 18/18 Dimethicone at a | |
| | weight ratio of 3:1 | |
| Locron L (ACH solution | Aluminum chlorohydrate | Clariant |
| 50%) | | |
| Plantaren ® 1200 | LAURYL GLUCOSIDE, | BASF |
| | approx. 50% AS | |
| Plantaren ® 2000 | DECYL GLUCOSIDE, | BASF |
| | approx. 50% AS | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product, comprising:
i) a package, selected from a pump spray container, a squeeze container, and a spray can containing at least one propellant, and
ii) a perspiration-inhibiting cosmetic agent contained therein for spray application, containing, in a cosmetically compatible carrier,
a) at least one perspiration-inhibiting zirconium-free aluminum salt in a total amount of 2 to 40 wt % wherein the specifications in wt. % refer to the total weight of the active substance (USP), free of water of crystallization and free of ligands, in the perspiration-inhibiting cosmetic agent without taking into account an optionally present propellant, and additionally
b) methanesulphonic acid of the following formula (MS-1) 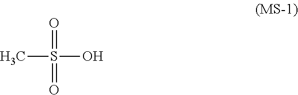
or at least one physiologically compatible salt of methanesulphonic acid.

2. The cosmetic product of claim 1, wherein the at least one perspiration-inhibiting zirconium-free aluminum salt comprises 8 to 35 wt. % of the perspiration-inhibiting cosmetic agent without taking into account an optionally present propellant.

3. The cosmetic product of claim 1, wherein the at least one perspiration-inhibiting zirconium-free aluminum salt comprises 10 to 28 wt. % of the perspiration-inhibiting cosmetic agent without taking into account an optionally present propellant.

4. The cosmetic product of claim 1, wherein the at least one physiologically compatible salt of methanesulphonic acid of formula MS-1 is a salt selected from the sodium, potassium, magnesium, calcium, manganese, zinc, and aluminum salts.

5. The cosmetic product of claim 1, wherein the methanesulphonic acid and/or at least one physiologically compatible salt of methanesulphonic acid comprises, 0.05 to 5 wt. % of the perspiration-inhibiting cosmetic agent without taking into account an optionally present propellant.

6. The cosmetic product of claim 1, wherein the methanesulphonic acid and/or at least one physiologically compatible salt of methanesulphonic acid comprises, 0.5 wt % to 2.5 wt % of the perspiration-inhibiting cosmetic agent without taking into account an optionally present propellant.

7. The cosmetic product according to claim 1, wherein the product further comprises at least one cosmetic oil that is not an odorous substance and not an essential oil.

8. The cosmetic product according to claim 1, wherein the perspiration-inhibiting cosmetic agent includes zero to a maximum of 10 wt. % free water, based on the weight of the perspiration-inhibiting cosmetic agent without taking into account an optionally present propellant.

9. The cosmetic product according to claim 1, wherein the perspiration-inhibiting cosmetic agent includes free water in a total amount of 15 to 96 wt. %, based on the total weight of the perspiration-inhibiting cosmetic agent without taking into account an optionally present propellant.

10. The cosmetic product according to claim 1, wherein the perspiration-inhibiting cosmetic agent includes free water in a total amount of 40 to 60 wt. %, based on the total weight of the perspiration-inhibiting cosmetic agent without taking into account an optionally present propellant.

11. The cosmetic product according to claim 1, wherein the characterized in that the perspiration-inhibiting cosmetic agent further comprises at least one emulsifying agent and/or at least one surfactant.

12. The cosmetic product according to claim 1, wherein the perspiration-inhibiting cosmetic agent is present as a water-in-oil emulsion.

13. The cosmetic product according to claim 1, wherein the perspiration-inhibiting cosmetic agent is present as an oil-in-water emulsion.

14. The cosmetic agent according to claim 1, wherein the perspiration-inhibiting cosmetic agent is present as an aqueous solution.

15. The cosmetic agent according to claim 14, wherein the aqueous solution include ethanol.

16. The cosmetic product according to claim 1, wherein the perspiration-inhibiting cosmetic agent is present as a suspension of the undissolved antiperspirant active substance in an oil.

17. Method for preventing and/or reducing textile discolorations and/or textile stains, wherein the method comprises the following method steps:
(a) producing a perspiration-inhibiting cosmetic agent by mixing at least one perspiration-inhibiting zirconium-free aluminum salt in a total amount of 2 to 40 wt. %, wherein the specifications in wt. % refer to the total weight of the active substance (USP), free of water of crystallization and free of ligands, in the perspiration-inhibiting cosmetic agent, with a cosmetically compatible carrier and with methanesulphonic acid of the following formula (MS-1) 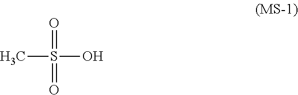
or at least one physiologically compatible salt of methanesulphonic acid, wherein methanesulphonic acid or the salt(s) thereof is/are included in a total amount of 0.05 to 5 wt. %, wherein the specifications in wt. % each refer to the total weight of the propellant-free agent;
(b) filling of the agent in a packaging selected from a pump spray container, a squeeze container, and a spray can containing at least one propellant;
(c) applying the perspiration-inhibiting cosmetic agent to skin surface of a subject;
(d) wearing a textile garment over the treated skin; and
(e) washing the textile garment,
wherein no, or reduced, textile discolorations and/or textile stains occur after washing.
